(19) United States
(12) Patent Application Publication
(10) Pub. No.: US 2022/0378711 A1
(43) Pub. Date: Dec. 1, 2022

(54) PH-Responsive Nanoparticles for Treating Cancer

(71) Applicant: United States Government as represented by the Department of Veterans Affairs, Washington, DC (US)

(72) Inventors: Sushanta K. Banerjee, Kansas City, MO (US); Snigdha Banerjee, Kansas City, MO (US); Mohiuddin Quadir, Fargo, ND (US); Priyanka Ray, Fargo, ND (US)

(21) Appl. No.: 17/534,427

(22) Filed: Nov. 23, 2021

Related U.S. Application Data
(60) Provisional application No. 63/187,151, filed on May 11, 2021.

(51) Int. Cl. A61K 9/51; A61K 31/506; A61K 31/7068; A61K 47/42; A61K 9/48

(52) U.S. Cl. CPC A61K 9/5123; A61K 31/506; A61K 31/7068; A61K 47/42; A61K 9/4816

(57) ABSTRACT

Disclosed are nanoparticles comprising a polymer carrier formed from a poly(ethylene glycol) and a poly(carbonate) copolymer which encapsulates a cargo entity such as an ERK inhibitor and/or chemotherapeutic agent. Therapeutic uses of the nanoparticles are also described, including methods of treating a cancer such as pancreatic cancer.

### CROSS-REFERENCE TO RELATED APPLICATIONS

This application claims priority to U.S. Provisional Application No. 63/187,151, filed May 11, 2021, which is incorporated into this application in its entirety.

### BACKGROUND

Pancreatic ductal adenocarcinoma (PDAC), the most common form of pancreatic cancer, is the third leading cause of cancer-related death in the United States and a predicted second leading cause of cancer-related death by 2030. The Surveillance, Epidemiology and End Results (SEER) database estimates an overall five-year survival rate of about 8%, the lowest of all solid cancer types. The American Cancer Society estimates that in 2021, about 60,000 people will be diagnosed with PDAC, and about 48,000 will die of this disease. Primary causes for these dismal prognoses are the lack of early detection methods, the lack of novel therapeutic candidates, limited treatment modalities, and drug resistance.

Many existing chemotherapies have a sub-optimal effect due to weak cellular uptake attributable to poor penetration into the hypo-vascularized and dense tumor stroma, known as desmoplasia, that creates a barrier for drug delivery and causes drug resistance Desmoplasia, an interaction upshot of cancer stem cells (CSCs) and fibroblast cells in the tumor microenvironment, leads to hypoxia, promotes glycolysis and lactic acid formation, and causes acidosis (low pH) in the tumor microenvironment. In PDAC, for instance, early hypoxia triggers remodeling of the extracellular matrix acidification, epithelial-to-mesenchymal transition (EMT), cell survival, metastasis, the formation of CSCs/TICs, and significant resistance to chemo- and radiotherapy. In addition, hypoxic conditions trigger glycolytic switch in tumor cells, resulting in the cytosol and eventually extracellular matrix (ECM) acidification. Hypoxia, enhanced glucose uptake, and carbonic anhydrase activity on cancer cell surfaces also contribute to creating such acidic extracellular milieu favoring tumor growth, invasion, and the emergence of resistant metastatic phenotypes of PDAC.

In this hostile microenvironment of tumors, some cancer cells adapt to survive and gain functions to promote metastasis to the distant organs and develop drug resistance capacity. A need exists in the art for new therapies capable of delivering effective chemotherapeutic agents to pancreatic cancer cells in the hostile microenvironment of tumors while minimizing unwanted effects on healthy cells. This need is met by the following disclosure.

### SUMMARY

This disclosure relates to a composition comprising a nanoparticle having a core comprising an inhibitor of extracellular signal-regulated kinase 1 or 2 (ERK1 or ERK2) and a polymer carrier, wherein the polymer carrier encapsulates the inhibitor, wherein the polymer carrier is a copolymer of poly(ethylene glycol) (PEG) and poly(carbonate), and wherein the copolymer comprises a pH-responsive ligand that facilitates release of the inhibitor from the nanoparticle when the nanoparticle is present in an acidic or hypoxic environment.

Also disclosed is a method of treating a pancreatic cancer in a subject in need thereof, comprising administering to the subject a composition comprising: (a) a nanoparticle having a core comprising an inhibitor of extracellular signal-regulated kinase 1 or 2 (ERK1 or ERK2) and a polymer carrier, wherein the polymer carrier encapsulates the inhibitor, wherein the polymer carrier is a copolymer of poly(ethylene glycol) (PEG) and poly(carbonate), and wherein the polymer carrier comprises a pH-responsive ligand that facilitates release of the inhibitor from the nanoparticle when the nanoparticle is present in an acidic or hypoxic environment; and (b) a chemotherapeutic agent.

### BRIEF DESCRIPTION OF THE DRAWINGS

The foregoing summary, as well as the following description of the disclosure, is better understood when read in conjunction with the appended drawings. For the purpose of illustrating the disclosure, the drawings illustrate some, but not all, alternative embodiments. This disclosure is not limited to the precise arrangements and instrumentalities shown. The following figures, which are incorporated into and constitute part of the specification, assist in explaining the principles of the disclosure.

FIG. 1 is a schematic showing the general mechanism of action of one embodiment in which both an ERK inhibitor and a chemotherapeutic agent is locally delivered to a cancerous tumor such as a pancreatic cancerous tumor.

FIG. 2 is a diagrammatic illustration of spatioselective activation and synthetic route of GEM and ERKi-loaded, pH-responsive nanoparticles. The concept of the pH-responsive nanoparticle for the enrichment of ERKi and GEM inside cancer cells is illustrated.

FIG. 3A is a bar graph showing the particle size of nanoparticles prepared from pH-responsive PEG-DB systems in 10% serum as obtained from DLS. When particles were made from pH-irresponsive PEG-HX systems, the pH-dependent assembly was not identifiable. Data represent the mean±SD of 3 experiments; ns, nonsignificant.

FIG. 3B is a plot showing time-dependent particle size and size distribution of drug carriers composed of PEG-DB systems, which are stable at systemic pH in the presence of 10% plasma without significant loss of stability. The increase in particle size over time is most likely due to the deposition of protein corona on the nanoparticle surface. The number (%) in the y-axis indicates the number-average particle size.

FIG. 3C is a histogram plot showing particle size and size distribution when PEG-DB systems were loaded with ERKi or GEM.

FIG. 4A is a time-dependent plot showing drug release kinetics from pH-responsive nanoparticles in microenvironment-mimicking pH conditions: cumulative percent release ERKi from pH-responsive nanoparticles at pH 4.5 and 7.4 in 10% serum-containing media, compared to release of the drug from pH-nonresponsive systems.

FIG. 4B is a time-dependent plot showing the corresponding release profile for GEM encapsulated in the copolymeric block nanoparticle at pH 4.5 and pH 7.4 in the presence of 10% serum, compared with drug release from a pH-nonresponsive system. Error bars represent standard deviations when n=3.

FIG. 4C is a time-dependent plot showing the cumulative percentage release of GEM and ERKi from a 1:1 v/v mixture of drug-encapsulated, pH-responsive nanoparticles composed of PEG-DB (4) at two different pH environments. Error bars represent the standard deviation of the average of three measurements (n=3).

FIG. 5A is a plot showing the effect of free ERKi on MIA PaCa-2 cell viability. Error bars represent the mean±SD when n=6.

FIG. 5B is a plot showing the effect of free GEM on MIA PaCa-2 viability. Error bars represent the mean±SD when n=6.

FIG. 5C is a plot showing the effect of pH-responsive nanoparticle encapsulated ERKi on MIA PaCa-2 cell viability. Error bars represent the mean±SD when n=6.

FIG. 5D is a plot showing the effect of pH-responsive nanoparticle encapsulated GEM on MIA PaCa-2 cell viability. Error bars represent the mean±SD when n=6.

FIG. 5E is a plot showing the detection of synergistic cytotoxic activity of combined unencapsulated ERKi and GEM for MIA PaCa-2. When plotted in CompuSyn, the synergy was maintained in multiple ratios of concentrations as indicated by the data points below the CI. Error bars represent the standard deviation of the average of three measurements (n=6).

FIG. 5F is a plot showing the detection of synergistic cytotoxic activity of combined encapsulated ERKi and GEM for MIA PaCa-2. When plotted in CompuSyn, the synergy was maintained in multiple ratios of concentrations as indicated by the data points below the CI. Error bars represent the standard deviation of the average of three measurements (n=6).

FIG. 5G is a plot showing the effect of free ERKi on Panc-1 cell viability. Error bars represent the mean±SD when n=6.

FIG. 5H is a plot showing the effect of free GEM on Panc-1 cell viability. Error bars represent the mean±SD when n=6.

FIG. 5I is a plot showing the effect of pH-responsive nanoparticle encapsulated ERKi on Panc-1 cell viability. Error bars represent the mean±SD when n=6.

FIG. 5J is a plot showing the effect of pH-responsive nanoparticle encapsulated GEM on Panc-1 cell viability. Error bars represent the mean±SD when n=6.

FIG. 5K is a plot showing the detection of synergistic cytotoxic activity of combined unencapsulated ERKi and GEM for Panc-1. When plotted in CompuSyn, the synergy was maintained in multiple ratios of concentrations as indicated by the data points below the CI.

FIG. 5L is a plot showing the detection of synergistic cytotoxic activity of combined encapsulated ERKi and GEM for Panc-1. When plotted in CompuSyn, the synergy was maintained in multiple ratios of concentrations as indicated by the data points below the CI. Error bars represent the standard deviation of the average of three measurements (n=6).

FIG. 6 is a violin plot showing the fluorescence intensity for pH-responsive nanoparticles as measured using NIS Elements BR software. MIA PaCa-2 cells were exposed to Alexa Fluor 647-tagged pH-responsive nanoparticles for two time points, and the cellular internalization of pH NPs was determined using a confocal microscope.

FIG. 7A is a plot of the p-ERK/GAPDH ratio as a function of ERKi concentration as measured by Western Blotting, which shows the differential effect of free ERKi on ERK activation in PDAC cells. MIA PaCa-2 PDAC cells were treated with increasing concentrations (0.1-100 nM) of SCH 772984, either free or encapsulated in nanoparticles composed of PEG-DB. Western blotting for phosphorylated or total ERK (p-ERK, ERK) and phosphorylated and total RSK (p-RSK and RSK) was performed. GAPDH is used as an internal control. ERKi is the ERK inhibitor, SCH 772984. VC, vehicle control (2% DMSO). ns, nonsignificant. NP alone, drug-free NPs.

FIG. 7B is a plot of the ERK/GAPDH ratio as a function of ERKi concentration as measured by Western Blotting, which shows the differential effect of free ERKi on ERK activation in PDAC cells. MIA PaCa-2 PDAC cells were treated with increasing concentrations (0.1-100 nM) of SCH 772984, either free or encapsulated in nanoparticles composed of PEG-DB. Western blotting for phosphorylated or total ERK (p-ERK, ERK) and phosphorylated and total RSK (p-RSK and RSK) was performed. GAPDH is used as an internal control. ERKi is the ERK inhibitor, SCH 772984. VC, vehicle control (2% DMSO). ns, nonsignificant. NP alone, drug-free NPs.

FIG. 7C is a plot of the p-RSK/GAPDH ratio as a function of ERKi concentration as measured by Western Blotting, which shows the differential effect of free ERKi on ERK activation in PDAC cells. MIA PaCa-2 PDAC cells were treated with increasing concentrations (0.1-100 nM) of SCH 772984, either free or encapsulated in nanoparticles composed of PEG-DB. Western blotting for phosphorylated or total ERK (p-ERK, ERK) and phosphorylated and total RSK (p-RSK and RSK) was performed. GAPDH is used as an internal control. ERKi is the ERK inhibitor, SCH 772984. VC, vehicle control (2% DMSO). ns, nonsignificant. NP alone, drug-free NPs.

FIG. 7D is a plot of the RSK/GAPDH ratio as a function of ERKi concentration as measured by Western Blotting, which shows the differential effect of free ERKi on ERK activation in PDAC cells. MIA PaCa-2 PDAC cells were treated with increasing concentrations (0.1-100 nM) of SCH 772984, either free or encapsulated in nanoparticles composed of PEG-DB. Western blotting for phosphorylated or total ERK (p-ERK, ERK) and phosphorylated and total RSK (p-RSK and RSK) was performed. GAPDH is used as an internal control. ERKi is the ERK inhibitor, SCH 772984. VC, vehicle control (2% DMSO). ns, nonsignificant. NP alone, drug-free NPs.

FIG. 7E is a plot of the p-ERK/GAPDH ratio as a function of ERKi concentration as measured by Western Blotting, which shows the differential effect of ERKi-pH NPs on ERK activation in PDAC cells. MIA PaCa-2 PDAC cells were treated with increasing concentrations (0.1-100 nM) of SCH 772984, either free or encapsulated in nanoparticles composed of PEG-DB. Western blotting for phosphorylated or total ERK (p-ERK, ERK) and phosphorylated and total RSK (p-RSK and RSK) was performed. GAPDH is used as an internal control. ERKi is the ERK inhibitor, SCH 772984. VC, vehicle control (2% DMSO). ns, nonsignificant. NP alone, drug-free NPs.

FIG. 7F is a plot of the ERK/GAPDH ratio as a function of ERKi concentration as measured by Western Blotting, which shows the differential effect of ERKi-pH NPs on ERK activation in PDAC cells. MIA PaCa-2 PDAC cells were treated with increasing concentrations (0.1-100 nM) of SCH 772984, either free or encapsulated in nanoparticles composed of PEG-DB. Western blotting for phosphorylated or total ERK (p-ERK, ERK) and phosphorylated and total RSK (p-RSK and RSK) was performed. GAPDH is used as an internal control. ERKi is the ERK inhibitor, SCH 772984. VC, vehicle control (2% DMSO). ns, nonsignificant. NP alone, drug-free NPs.

FIG. 7G is a plot of the p-RSK/GAPDH ratio as a function of ERKi concentration as measured by Western Blotting, which shows the differential effect of ERKi-pH NPs on ERK activation in PDAC cells. MIA PaCa-2 PDAC cells were treated with increasing concentrations (0.1-100 nM) of SCH 772984, either free or encapsulated in nanoparticles composed of PEG-DB. Western blotting for phosphorylated or total ERK (p-ERK, ERK) and phosphorylated and total RSK (p-RSK and RSK) was performed. GAPDH is used as an internal control. ERKi is the ERK inhibitor, SCH 772984. VC, vehicle control (2% DMSO). ns, nonsignificant. NP alone, drug-free NPs.

FIG. 7H is a plot of the RSK/GAPDH ratio as a function of ERKi concentration as measured by Western Blotting, which shows the differential effect of ERKi-pH NPs on ERK activation in PDAC cells. MIA PaCa-2 PDAC cells were treated with increasing concentrations (0.1-100 nM) of SCH 772984, either free or encapsulated in nanoparticles composed of PEG-DB. Western blotting for phosphorylated or total ERK (p-ERK, ERK) and phosphorylated and total RSK (p-RSK and RSK) was performed. GAPDH is used as an internal control. ERKi is the ERK inhibitor, SCH 772984. VC, vehicle control (2% DMSO). ns, nonsignificant. NP alone, drug-free NPs.

FIG. 8A is a plot that shows the effect of encapsulated ERKi and GEM on PDAC clonogenic survival. MIA PaCa-2 cells (250 cells/well) were treated with the DMSO control, NP alone, 10 nM free/encapsulated ERKi, 100 nM free/encapsulated GEM, or 10 nM free/encapsulated ERKi+100 nM free/encapsulated GEM for 12 days before colonies were counted. Three replicate experiments were performed. The survival fractions of cells in plates were treated with DMSO or NP alone as untreated controls. Colonies were counted by the Colony Doc-It imaging station using Colony Doc-It imaging software. Data represent the mean±SD of three experiments. ns, nonsignificant and **p<0.05, ANOVA and student t-test.

FIG. 8B is a plot that shows the effect of encapsulated ERKi and GEM on PDAC clonogenic survival. Panc-1 cells (250 cells/well) were treated with the DMSO control, NP alone, 10 nM free/encapsulated ERKi, 100 nM free/encapsulated GEM, or 10 nM free/encapsulated ERKi+100 nM free/encapsulated GEM for 12 days before colonies were counted. Three replicate experiments were performed. The survival fractions of cells in plates were treated with DMSO or NP alone as untreated controls. Colonies were counted by the Colony Doc-It imaging station using Colony Doc-It imaging software. Data represent the mean±SD of three experiments. ns, nonsignificant and **p<0.05, ANOVA and student t-test.

FIG. 9A is a bar graph illustrating drug concentrations in MIA PaCa-2 tumor xenograft samples after 48 h of injection, N=3. F, free; N, NPs.

FIG. 9B is a plot that shows the therapeutic efficacy of free and pH-nanoparticle-encapsulated ERKi and GEM alone or combination treatment on the primary subcutaneous tumor growth in nude mice. Data represent mean primary tumor volumes ±SEM. N=4-7 mice/group. *p<0.001 and **P<0.002.

FIG. 9C is a bar graph that shows the treatment effect on tumor cell proliferation measured by calculating Ki67-positive cells in Ki67 immunohistochemically stained sections (five sections/tumor) of three animals: untreated, free drug combination, and encapsulated drug combination. The bar graph represents the percent of Ki67-positive cells. Data represent mean±SD. Scale, 100 μm.

FIG. 9D is a violin plot that represents the staining intensities of p-ERK in different samples of tumor xenografts measured by NIS Elements BR software.

FIG. 10A is a distribution plot showing the stability of particles over 30 days in terms of particle size and dispersity.

FIG. 10B is a plot showing the evolution of Zeta potential (surface charge) of particles over 30 days.

FIG. 11 is a plot showing cumulative drug release profile in PBS buffer and PBS with 10% added Lysis buffer solution. Korsmeyer-Peppas model to demonstrate the kinetics of drug release for drug release mechanism in PBS buffer and PBS with 10% added Lysis buffer solution.

FIG. 12A is a histogram showing representative results at 0 h for the cellular uptake study of dye labeled pH/hypoxia-responsive polymer encapsulated ERK-inhibitor on MiaPaca-2 cell line for Alexa Fluor 647.

FIG. 12B is a histogram showing representative results at 12 h for the cellular uptake study of dye labeled pH/hypoxia-responsive polymer encapsulated ERK-inhibitor on MiaPaca-2 cell line for DAPI.

FIG. 12C is a histogram showing representative results at 24 h for the cellular uptake study of dye labeled pH/hypoxia-responsive polymer encapsulated ERK-inhibitor on MiaPaca-2 cell line for Phalloidin.

FIG. 12D is a histogram showing representative merged results for the cellular uptake study of dye labeled pH/hypoxia-responsive polymer encapsulated ERK-inhibitor on MiaPaca-2 cell line.

FIG. 13 is a bar graph showing representative results for wound assay healing assay on MiaPaca-2. The bar graph represents the open wound percentage before and after treatment with different drug combinations.

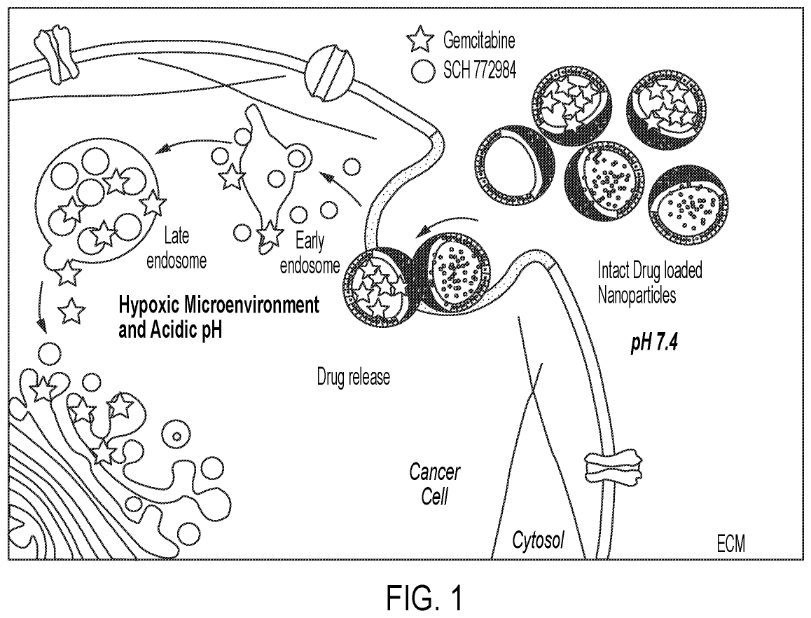

### DETAILED DESCRIPTION

### A. Definitions

“Nanoparticle” means a particle formed from a disclosed copolymer that has an average particle size of less than about 500 nm, as measured by dynamic light scattering (DLS), e.g., less than about 400 nm, less than about 300 nm, less than about 200 nm, less than about 190 nm, or from about 100 to about 190 nm or from about 100 to about 150 nm. In some aspects, average particle size, when measured by DLS, can represent the hydrodynamic diameter of the particle. DLS measurements typically have a margin of error around ±10%.

A “polycarbonate” refers to a polymer or portion of a polymer having the following functional group:

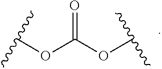 [SMILES: *OC(=O)O* ; "."]

When a polycarbonate is present in a copolymer comprising polyethylene glycol, the polycarbonate residue may be denominated in a formula as follows, which does not necessarily show the entire carbonate functional group in a discrete repeating unit:

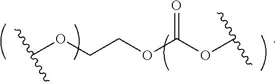

“Polyethylene glycol” or “PEG” refers to a polymer or portion of a polymer having the following repeating unit:

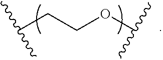

A “copolymer of poly(ethylene glycol) (PEG) and a poly(carbonate)” refers to a copolymer having the following general structure:

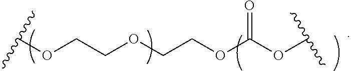

A “pH-responsive ligand that facilitates release” of a cargo entity (such as an ERK inhibitor or chemotherapeutic agent) encapsulated by a nanoparticle refers to a ligand that includes at least one amine group (i.e., a primary, secondary, or tertiary amine) that imparts a pKₐ of less than a plasma pH of 7.4 to the pH-responsive ligand or polymer carrier comprising the pH-responsive ligand, such that when the nanoparticle is present in an environment having a pH less than the plasma pH of 7.4 (and/or a hypoxic environment), at least a portion of the amine groups become protonated, leading to the destabilization of the self-assembled nanoparticle and release of the encapsulated cargo entity. In some aspects, a “pH-responsive ligand” refers to a ligand that imparts a pKₐ to the copolymer as a whole, as measured by the pH at the half-equivalence (inflection) point of a titration curve, where at least 50% of the amine groups located on the copolymer are in protonated form, that is less than 7.4, e.g., less than or equal to 7.3, less than or equal to 7.2, less than or equal to 7.1, less than or equal to 7.0, less than or equal to 6.9, less than or equal to 6.8, less than or equal to 6.7, less than or equal to 6.6, less than or equal to 6.5, less than or equal to 6.4, less than or equal to 6.3, less than or equal to 6.2, less than or equal to 6.1, less than or equal to 6.0, less than or equal to 5.9, less than or equal to 5.8, less than or equal to 5.7, less than or equal to 5.6, less than or equal to 5.5, less than or equal to 5.4, less than or equal to 5.3, less than or equal to 5.2, less than or equal to 5.1, less than or equal to 5.0, less than or equal to 4.9, less than or equal to 4.8, less than or equal to 4.7, less than or equal to 4.6, less than or equal to 4.5, less than or equal to 4.4, less than or equal to 4.3, less than or equal to 4.2, less than or equal to 4.1, or less than or equal to 4.0.

An “acidic environment” means an environment (e.g., an environment in a subject in or around a tumor such as a pancreatic cancer tumor, or a PDAC tumor in some aspects) having a pH of less than 7, e.g., a pH of less than or equal to 6.9, a pH of less than or equal to 6.8, a pH of less than or equal to 6.7, a pH of less than or equal to 6.6, a pH of less than or equal to 6.5, or pH of less than or equal to 6.4, a pH of less than or equal to 6.3, a pH of less than or equal to 6.2, a pH of less than or equal to 6.1, a pH of less than or equal to 6.0, a pH of less than or equal to 5.9, a pH of less than or equal to 5.8, a pH of less than or equal to 5.7, a pH of less than or equal to 5.6, a pH of less than or equal to 5.6, a pH of less than or equal to 5.5, a pH of less than or equal to 5.4, a pH of less than or equal to 5.3, a pH of less than or equal to 5.2, a pH of less than or equal to 5.1, or a pH of less than or equal to 5.0.

A “hypoxic environment” means an environment (e.g., an environment in a subject in or around a tumor such as a pancreatic cancer tumor, or a PDAC tumor in some aspects) exhibiting a partial pressure of oxygen (pO₂) of less than 80 mm Hg, e.g., an environment exhibiting a pO₂ of less than or equal to 75 mm Hg, less than or equal to 70 mm Hg, less than or equal to 65 mm Hg, less than or equal to 60 mm Hg, less than or equal to 55 mm Hg, less than or equal to 50 mm or equal to Hg, less than or equal to 45 mm Hg, less than or equal to 40 mm Hg, less than or equal to 35 mm Hg, less than or equal to 35 mm Hg, less than or equal to 30 mm Hg, less than or equal to 25 mm Hg, less than or equal to 24 mm Hg, less than or equal to 23 mm Hg, less than or equal to 22 mm Hg, less than or equal to 21 mm Hg, less than or equal to 20 mm Hg, less than or equal to 19 mm Hg, less than or equal to 18 mm Hg, less than or equal to 17 mm Hg, less than or equal to 16 mm Hg, less than or equal to 15 mm Hg, less than or equal to 14 mm Hg, less than or equal to 13 mm Hg, less than or equal to 12 mm Hg, less than or equal to 11 mm Hg, less than or equal to 10 mm Hg, less than or equal to 9 mm Hg, less than or equal to 8 mm Hg, less than or equal to 7 mm Hg, less than or equal to 6 mm Hg, less than or equal to 5 mm Hg, less than or equal to 4 mm Hg, less than or equal to 3 mm Hg, less than or equal to 2 mm Hg, less than or equal to 1 mm Hg, or 0 mm Hg.

An “acidic or hypoxic environment” is an environment (e.g., an environment in a subject in or around a tumor such as a pancreatic cancer tumor, or a PDAC tumor in some aspects) that is acidic, hypoxic, or both.

“Ligand” means a moiety capable of covalently or otherwise bonding to the described copolymer.

A “ligand that targets a pancreatic cancer cell” means a ligand that is covalently or otherwise attached to the described copolymer, which is capable of penetrating a pancreatic cancer cell, specifically by penetrating desmoplastic tumor tissue, a dense layer of fibrotic tissue that ordinarily resists penetration by therapeutic agents. In one aspect, but without being bound by any theory, the ligand that targets the pancreatic cancer cell is capable of penetrating a pancreatic cancer tumor (e.g., a PDAC tumor) via a molecular pathway mediated by β5 integrin, a protein that is produced by carcinoma-associated fibroblasts (CAFs).

“Small molecule” means an organic molecule having a molecular weight less than 10,000 g/mole, e.g., less than 5,000 g/mole, less than 4,000 g/mole, less than 3,000 g/mole, less than 2,000 g/mole, less than 1,000 g/mole, less than 900 g/mole, less than 800 g/mole, less than 700 g/mole, or less than 600 g/mol.

“C₁-C₁₂ alkyl” means a branched or unbranched saturated hydrocarbon having 1 to 12 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, or dodecyl. The alkyl group can be a C₁ alkyl, C₁-C₂ alkyl, C₁-C₃ alkyl, C₁-C₄ alkyl, C₁-C₅ alkyl, C₁-C₆ alkyl, C₁-C₇ alkyl, C₁-C₈ alkyl, C₁-C₉ alkyl, C₁-C₁₀ alkyl, C₁-C₁₁ alkyl, or C₁-C₁₂ alkyl.

“C₁-C₁₂ alkylamino” means a branched or unbranched saturated hydrocarbon having 1 to 12 carbon atoms and at least one amino group (e.g., —NH₂, —NHR, where R is a non-hydrogen substituent, or —NRR′, where R and R′ are non-hydrogen substituents). Representative examples include methylamino, ethylamino, propylamino, isopropylamino, butylamino, isobutylamino, (sec-butyl)amino, (tert-butyl)amino, pentylamino, isopentylamino, (tert-pentyl)amino, and hexylamino, and the like. When an R group comprises a C₁-C₁₂ alkylamino, the alkylamino group is not necessarily connected to the substrate to which the R group is attached through the amino group, i.e., the alkylamino group can be connected at a point on the alkyl chain.

“C₂-C₁₂ dialkylamino” means a hydrocarbon having 1 to 12 carbon atoms and at least one amino group, i.e., a substituent that includes the formula —N(-alkyl)₂. Representative examples include dimethylamino, diethylamino, dipropylamino, diisopropylamino, dibutylamino, diisobutylamino, di(sec-butyl)amino, di(tert-butyl)amino, dipentylamino, diisopentylamino, di(tert-pentyl)amino, dihexylamino, N-ethyl-N-methylamino, N-methyl-N-propylamino, and N-ethyl-N-propylamino. As with alkylamino groups, when an R group comprises a C₁-C₁₂ dialkylamino, the dialkylamino group is not necessarily connected to the substrate to which the R group is attached through the amino group, i.e., the dialkylamino group can be connected at a point on the alkyl chain.

“C₃-C₁₂ cycloalkyl” means a non-aromatic carbon-based ring having 3 to 12 carbons. Examples of cycloalkyl groups include cyclopropyl, cyclobutyl, cyclopentyl, cyclohexyl, and norbornyl.

“C₂-C₁₂ heterocycloalkyl” is a non-aromatic carbon-based ring having 2 to 12 carbons. At least one member of the heterocycloalkyl ring is a heteroatom such as nitrogen, oxygen, sulfur, or phosphorous.

Any of the described therapeutic agents, including ERK inhibitors and chemotherapeutic agents, although shown or described in neutral form, can be present as an acceptable pharmaceutically acceptable salt. Gemcitabine, as one illustrative example, can be in the form of an acceptable salt such as gemcitabine hydrochloride.

“Subject” means any living subject including mammalian subjects such as a human.

“Administering” refers to any method of providing a therapeutic composition to a subject. Methods of administration include oral administration and parenteral administration, including injectable such as intravenous administration, intra-arterial administration, intramuscular administration, and subcutaneous administration. Administration can be continuous or intermittent.

When the term “about” precedes a numerical value, the numerical value can vary within ±10% unless specified otherwise.

### B. Compositions

The compositions are based on the inventors' discovery of a nanodelivery system that overcomes the desmoplastic barrier common in pancreatic cancer cells. The nanodelivery system can also facilitates release of therapeutic agents directly into the hypoxic or acidic environment of a tumor to effectively release the therapeutic agents locally, preventing or reducing cancer cell growth, while minimizing effects on healthy cells.

The compositions comprise a nanoparticle having a core comprising an inhibitor of extracellular signal-regulated kinase 1 or 2 (ERK1 or ERK2) (“ERK inhibitor”) and a polymer carrier. The polymer carrier encapsulates the inhibitor. In one aspect, the polymer carrier comprises a copolymer of poly(ethylene glycol) (PEG) and a poly(carbonate), which can include one or more pH-responsive ligands that facilitate release of the inhibitor from the nanoparticle when the nanoparticle is present in an acidic or hypoxic environment common with many pancreatic cancers including PDAC.

1. Nanoparticles

The nanoparticle of the composition is generally a carrier (delivery vehicle) for the ERK inhibitor, optionally a carrier (delivery vehicle) for the chemotherapeutic agent, or optionally a carrier (delivery vehicle) for both the ERK inhibitor and the chemotherapeutic agent. In other words, the composition can include individual nanoparticles encapsulating the ERK inhibitor, along with individual nanoparticles encapsulating the chemotherapeutic agent, or the ERK inhibitor and chemotherapeutic agent can be encapsulated by the same nanoparticles, or any combination comprising individually encapsulated ERK inhibitors and chemotherapeutic agents together with nanoparticles encapsulating both an ERK inhibitor and the chemotherapeutic agent.

In some aspects, however, the composition includes a nanoparticle encapsulating an ERK inhibitor and a free or unencapsulated chemotherapeutic agent. Alternatively, the composition can include a nanoparticle-encapsulated ERK inhibitor, and a treatment method can involve administering the nanoparticle composition together, concomitantly, or sequentially with the administration of a free or unencapsulated chemotherapeutic agent.

The polymer carrier generally comprises a copolymer of poly(ethylene glycol) (PEG) and a poly(carbonate). The copolymer can be a random copolymer of PEG and the poly(carbonate) but in some aspects the copolymer is a block copolymer featuring a PEG block and a block of the polycarbonate.

The molecular weight of the copolymer can vary. In some aspects, the copolymer has a number-average molecular weight (Ma) as determined by gel-permeation chromatography (GPC) ranging from about 1,000 Daltons to about 50,000 Daltons. In a further aspect, the Mₙ of the copolymer ranges from about 5,000 Daltons to about 50,000 Daltons, e.g., about 10,000 Daltons to about 50,000 Daltons, about 15,000 Daltons to about 50,000 Daltons, or about 20,000 Daltons to about 50,000 Daltons. In a still further aspect, the Mₙ of the copolymer ranges from about 20,000 Daltons to about 40,000 Daltons, e.g., about 20,000 Daltons, about 25,000 Daltons, about 30,000 Daltons, or about 45,000 Daltons. In some aspects, when the copolymer is a block copolymer, the PEG block has a molecular weight ranging from about 5,000 Daltons to about 20,000 Daltons, while the poly(carbonate) block has a molecular weight of about 15,000 Daltons. Specific non-limiting examples are shown below in Table 1.

TABLE 1
Exemplary Block Copolymer Molecular Weights

| PEG Block | Poly(carbonate) Block | Total |
|---|---|---|
| Molecular Weight | Molecular Weight | Molecular Weight |
| 5,000 Daltons | 15,000 Daltons | 20,000 Daltons |
| 10,000 Daltons | 15,000 Daltons | 25,000 Daltons |
| 15,000 Daltons | 15,000 Daltons | 30,000 Daltons |
| 20,000 Daltons | 15,000 Daltons | 35,000 Daltons |

In one aspect, the nanoparticle has an average particle size as measured by DLS of less than about 200 nm, e.g., 20-200 nm, 30-200 nm, 40-200 nm, 50-200 nm, 60-200 nm, 70-200 nm, 80-200 nm, 100-200 nm, 100-190 nm, 100-180 nm, or 100-150 nm.

In some aspects, the copolymer has a structure represented by the formula:

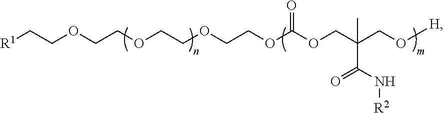

wherein R¹ is C₁-C₄ alkoxy or a ligand that targets a pancreatic cancer cell, R² is C₁-C₁₂ alkyl, C₁-C₁₂ alkyl, C₁-C₁₂ alkylamino, C₂-C₁₂ dialkylamino, C₃-C₁₂ cycloalkyl, or C₂-C₁₂ heterocycloalkyl, and n and m are independently integers ranging from 10 to 300, e.g., 15 to 300, 20 to 300, 50 to 300, 60 to 300, 80 to 300, or 100 to 300.

R¹ can in general be a number of suitable end groups or can be a ligand that targets a pancreatic cancer cell such as a PDAC cell. In one specific aspect, R¹ is —OCH₃, —OCH₂CH₃, —OCH₂CH₂CH₃, or —OCH₂CH₂CH₂CH₃. Alternatively, R¹ can be a ligand that targets a pancreatic cancer cell such as a PDAC cell. A variety of ligands are contemplated. In one aspects, the ligand comprises an arginine-glycine-aspartate (RGD) residue. In another aspect, the ligand comprises iRGD, a nine-amino acid peptide having the following structure:

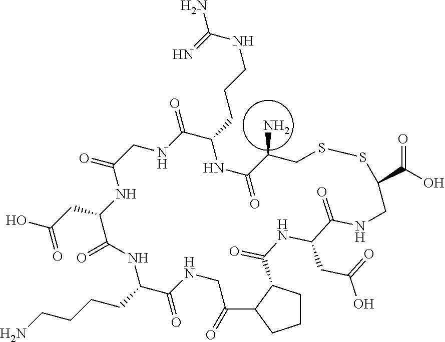 [SMILES: N=C(N)NCCC[C@@H]1NC(=O)[C@@H](N)CSS[C@@H](C(=O)O)CNC(=O)[C@H](CC(=O)O)NC(=O)[C@@H]2CCCC2C(=O)CNC(=O)[C@H](CCCCN)NC(=O)[C@H](CC(=O)O)NC(=O)CNC1=O]

iRGD can be conveniently reacted with the copolymer through a variety of sites, including the primary amino group on the cysteine residue circled in the structure above. For instance, through the use of Click chemistry, iRGD modified with a terminal alkyne group at the cysteine residue can be reacted with an azide functionality on an end group of the PEG chain, as described further in the Examples below. Thus, for example, R¹ can have a structure represented by either of the following two structures after attaching the iRGD ligand to the copolymer:

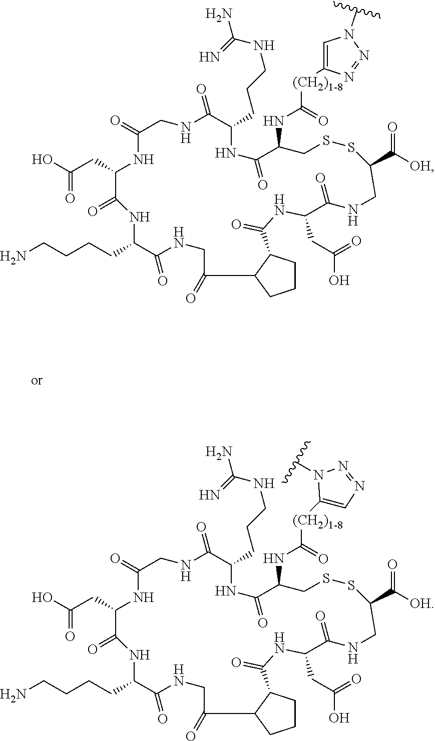 [SMILES: *n1cc(CC(=O)N[C@H]2CSS[C@@H](C(=O)O)CNC(=O)[C@H](CC(=O)O)NC(=O)[C@@H]3CCCC3C(=O)CNC(=O)[C@H](CCCCN)NC(=O)[C@H](CC(=O)O)NC(=O)CNC(=O)[C@H](CCCNC(=N)N)NC2=O)nn1 ; *n1nncc1CC(=O)N[C@H]1CSS[C@@H](C(=O)O)CNC(=O)[C@H](CC(=O)O)NC(=O)[C@@H]2CCCC2C(=O)CNC(=O)[C@H](CCCCN)NC(=O)[C@H](CC(=O)O)NC(=O)CNC(=O)[C@H](CCCNC(=N)N)NC1=O]

As shown in the structures above, the alkyl chain separating the cysteine residue from the copolymer can in general vary, e.g., it can have from between 1-8 carbon atoms. Specific examples include instances in which R¹ corresponds to one of the following structures:

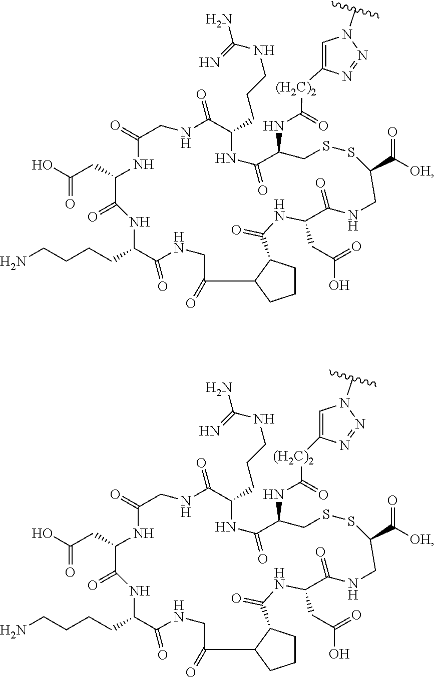

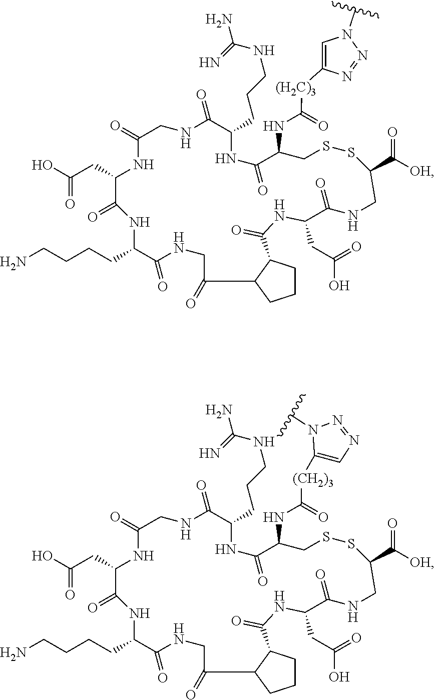

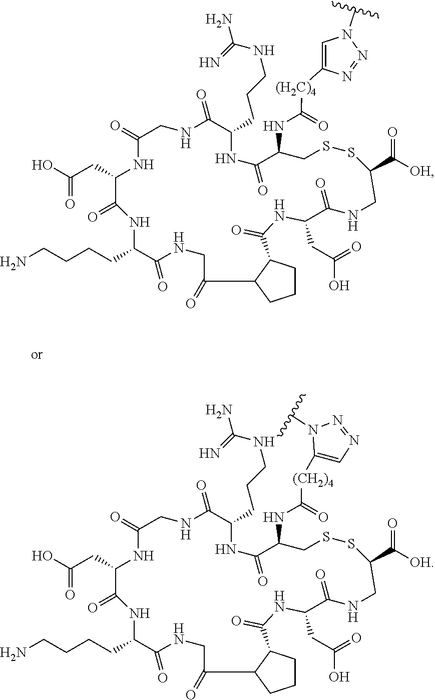

R² in general is a pH-responsive ligand that when present in an acidic or hypoxic environment (e.g., an acidic of hypoxic environment of a pancreatic cancer tumor such as a PDAC tumor), has at least one primary, secondary, or tertiary amine group that becomes protonated and thereby causes the destabilization of the self-assembled nanoparticle, allowing for the ERK inhibitor and/or the chemotherapeutic agent to release from the nanoparticle into the surrounding tissue. In some aspects, because the poly(carbonate) residue has an amide functionality that can impart a suitable pKₐ to achieve this purpose, R² can be C₁-C₁₂ alkyl or C₃-C₁₂ cycloalkyl. In further aspects, R² can comprise additional amine groups, e.g., R² can be C₁-C₁₂ alkylamino, C₂-C₁₂ dialkylamino or C₂-C₁₂ heterocycloalkyl, wherein the heterocycloalkyl group comprises in some aspects at least one nitrogen atom.

Specific non-limiting examples of R² groups include the following:

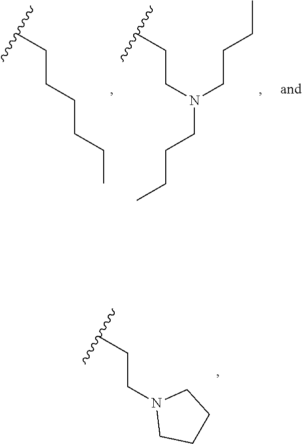

2. ERK Inhibitors

The extracellular signal-regulated kinases are one class of signaling kinases that are involved in conveying extracellular signals into cells and subcellular organelles. ERK1 and ERK2 are involved in regulating a wide range of activities and dysregulation of the ERK1/2 cascade is known to cause a variety of pathologies including neurodegenerative diseases, developmental diseases, diabetes, and cancer. The role of ERK1/2 in cancer is of special interest because activating mutations upstream of ERK1/2 in its signaling cascade are believed to be responsible for more than half of all cancers. Moreover, excessive ERK1/2 activity has also been found in cancers where the upstream components were not mutated, suggesting that ERK1/2 signaling plays a role in carcinogenesis even in cancers without mutational activations. The ERK pathway has also been shown to control tumor cell migration and invasion, and thus may be associated with metastasis.

In one aspect, the ERK inhibitor is a small molecule ERK inhibitor. In some specific aspects, the ERK inhibitor is BVD-523, FR 180204, MK-8353 (SCH900353), pluripotin, SCH772984, VX-1 le (ERK-1 le; TCS ERK 1 le), SL327, hypericin, purvalanol, PD173074, GW5074, BAY 43-9006, AG99, CAY10561, ISIS 5132, apigenin, SP600125, SU4984, SB203580, PD169316, K0947, GDC0994, and AG1478. Other inhibitors include, but are not limited to, chromone and flavone type inhibitors; PD 98059 (Runden E et al, J Neurosci 1998, 18(18) 7296-305); PD0325901 (Pfizer); Selumetinib, a selective MEK inhibitor (AstraZeneca/Array BioPharma, also known as AZD6244); ARRY-438162 (Array BioPharma); PD198306 (Pfizer); PD0325901 (Pfizer); AZD8330 (AstraZeneca/Array Biopharma, also called ARRY-424704); PD 184352 (Pfizer, also called Cl-1040); PD 184161 (Pfizer); a-[Amino[(4-aminophenyl)thio]methylene]-2-(trifluoromethyl)benzeneacetonitrile (SL327); 1,4-Diamino-2,3-dicyano-1,4-bis(2-aminophenylthio)butadiene; U0126 (Kohno & Pouyssegur (2003) Prog. Cell. Cyc. Res. 5: 219-224); GW 5074 (Santa Cruz Biotechnology); BAY 43-9006 (Bayer, Sorafenib); RO 09-2210 (Roche, Williams et al, Biochemistry. 1998 Jun. 30; 37(26):9579-85); FR 1 80204 (Ohori, M. et al. (2005) Biochem. Biophys. Res. Comm. 336: 357-363); 3-(2-aminoethyl)-5-))4-ethoxyphenyl)methylene)-2,4-thiazolidinedione (PKI-ERK-005) (Chen, F. et al. (2006) Bioorg. Med. Chem. 16:6281-6288. 171. Hancock, C N. et al. (2005) J. Med. Chem. 48: 4586-4595); CAY10561 (CAS 933786-58-4; Cayman Chemical); GSK 120212; RDEA1 19 (Ardea Biosciences); XL518; ARRY-704 (AstraZeneca); or any combination thereof.

In another specific aspect, the ERK inhibitor has a structure represented by the formula:

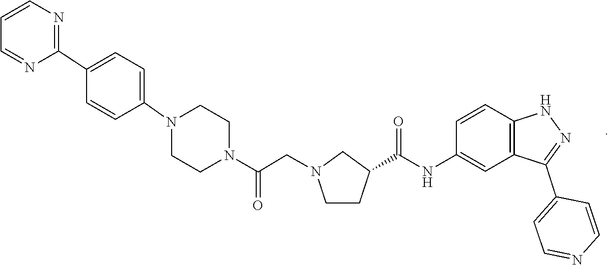 [SMILES: O=C(Nc1ccc2[nH]nc(-c3ccncc3)c2c1)[C@@H]1CCN(CC(=O)N2CCN(c3ccc(-c4ncccn4)cc3)CC2)C1 ; "."]

3. Chemotherapeutic Agents

In some aspects, the disclosed compositions further comprise a chemotherapeutic agent, either in free or unencapsulated form or in encapsulated form (e.g., encapsulated along with the ERK inhibitor in the same nanoparticle or separately encapsulated in other nanoparticles, which can in general be the same or different as the nanoparticles encapsulating the ERK inhibitor.

In one aspect, the chemotherapeutic agent is a nucleoside analog. In a further aspect, the nucleoside analog is a pyrimidine nucleoside prodrug. In a still further aspect, the chemotherapeutic agent is an analog of deoxycytidine nucleoside (2′-deoxy-2′,2′-difluorocytidine; dFdC).

In some aspects, the chemotherapeutic agent is gemcitabine (GEMZAR), 5-fluorouracil (5-FU), fluoropyrimidine, irinotecan (CAMPTOSAR), oxaliplatin (ELOXATIN), paclitaxel (TAXOL) or albumin-bound paclitaxel (ABRAXANE), capecitabine (XELODA), cisplatin, docetaxel (TAXOTERE), irinotecan liposome (ONIVYDE), or any combination thereof, including for example 5-FU/leucovorin/irinotecan/oxaliplatin (FOLFIRINOX). In one specific aspect, the chemotherapeutic agent is gemcitabine.

4. Pharmaceutical Compositions

In one aspect, the composition of nanoparticles and/or a separate composition comprising the chemotherapeutic agent can be in the form of an administrable pharmaceutical composition and thus can include other ingredients aside from those described above. An administrable composition can include the disclosed composition in combination with a pharmaceutically acceptable excipient, such as a preservative, buffer, saline, or phosphate buffered saline, depending on the route of administration. Suitable forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsiloxane), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A form suitable for injectable use can have a disclosed composition suspended in sterile saline solution for injection together with a preservative.

Pharmaceutical compositions can also comprise a suitable pharmaceutically acceptable carrier, such as sterile aqueous or nonaqueous solutions, dispersions, suspensions or emulsions, as well as sterile powders for reconstitution into sterile injectable solutions or dispersions prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate.

Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption.

Injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

### C. Treatment Methods

The disclosed nanoparticle compositions (including any of the specific compositions, nanoparticles, polymer carriers, therapeutic agents described above) can be administered to a subject in need of treatment either alone (e.g., when the nanoparticles comprise an ERK inhibitor, chemotherapeutic agent, or both, encapsulated in the same or different nanoparticles), or a nanoparticle composition comprising encapsulated ERK inhibitor can be administered separately from another chemotherapeutic composition (including any of the chemotherapeutic agents described above), which can be administered at the same or a different time relative to the nanoparticle composition comprising the encapsulated ERK inhibitor.

In one aspect, the method is a method of treating a pancreatic cancer (e.g., PDAC) in a subject in need thereof, comprising administering to the subject a composition comprising: (a) a nanoparticle having a core comprising an inhibitor of extracellular signal-regulated kinase 1 or 2 (ERK1 or ERK2) and a polymer carrier, wherein the polymer carrier encapsulates the inhibitor, wherein the polymer carrier is a copolymer of poly(ethylene glycol) (PEG) and a poly(carbonate), and wherein the copolymer comprises a pH-responsive ligand that facilitates release of the inhibitor from the nanoparticle when the nanoparticle is present in an acidic or hypoxic environment; and (b) a chemotherapeutic agent.

Any of the chemotherapeutic agents described above can be administered, either in encapsulated or unencapsulated form, or both. In one specific aspect, the chemotherapeutic agent is gemcitabine. In a further aspect, the chemotherapeutic agent is encapsulated by a polymer carrier of a nanoparticle having a core comprising the chemotherapeutic agent, wherein the polymer carrier is a copolymer of poly(ethylene glycol) (PEG) and poly(carbonate), and wherein the copolymer comprises a pH-responsive ligand that facilitates release of the inhibitor from the nanoparticle when the nanoparticle is present in an acidic or hypoxic environment.

In a further aspect, wherein the polymer carrier encapsulates the chemotherapeutic agent and the ERK inhibitor, and the pH-responsive ligand facilitates the release of the chemotherapeutic agent and the ERK inhibitor from the nanoparticle when the nanoparticle is present in an acidic or hypoxic environment.

In a further aspect, the method is a method of delivering an ERK inhibitor and/or a chemotherapeutic agent to a pancreatic cancer cell (e.g., a PDAC cell), comprising contacting the cell with a composition comprising: (a) a nanoparticle having a core comprising an inhibitor of extracellular signal-regulated kinase 1 or 2 (ERK1 or ERK2) and a polymer carrier, wherein the polymer carrier encapsulates the inhibitor, wherein the polymer carrier is a copolymer of poly(ethylene glycol) (PEG) and a poly(carbonate), and wherein the copolymer comprises a pH-responsive ligand that facilitates release of the inhibitor from the nanoparticle when the nanoparticle is present in an acidic or hypoxic environment; and (b) a chemotherapeutic agent, which can be in free or unencapsulated form or can be encapsulated by the same or different nanoparticles relative to the nanoparticles encapsulating the ERK inhibitor.

### D. Examples

The following examples further illustrate this disclosure. The scope of the disclosure and claims is not limited by the scope of the following examples.

1. Materials and Methods

Chemicals and Antibodies

All chemicals used in this study were obtained from Sigma-Aldrich (St. Louis, Mo., USA), and anhydrous solvents were purchased from VWR (Radnor, Pa., USA) and EMID Millipore. GEM and SCH 772984 were purchased from Cayman Chemical Inc (Ann Arbor, Mich., USA). Vybrant MTT cell proliferation assay kits were obtained from Thermo Fisher Scientific (Waltham, Mass., USA). Antibodies targeting ERK, phosphor-ERK, and j-actin were purchased from Cell Signaling Technology (Danvers, Mass., USA). ECL kits for Western blot were obtained from Abcam (Cambridge, Mass., USA). A Bruker 400 MHz spectrometer were used to record H NMR Spectra and utilize TMS as inner standard. ATR diamond tip was used to record I.R. Spectra Thermo Scientific Nicolet 8700 FTIR instrument. The size of the synthesized polymer was characterized by Gel Permeation Chromatographic using GPC (EcoSEC HLC) system. Polystyrene (Agilent EasiVial PS-H 4 ml) has been used as a standard (THF as the eluent with a flow rate of 0.35 mL per minute at 40° C.) using a differential R.I. detector. 1 mg/ml sample were injected into the system. Malvern (Malvern Z.S. 90) instrument were used to measure DLS. UV-vis spectrophotometer and a fluorescent Fluoro-Log 3 spectrophotometer were used to record UV-visual and fluorescence spectra respectively. MitoProbe JC-1 assay kit was purchased from Thermo Fisher Scientific (Waltham, Mass., USA).

Cell Lines and Culture Conditions

The MIA PaCa-2 and Panc-1 pancreatic cancer cell lines were obtained from the American Tissue Culture Type (ATCC, Manassas, Va., USA), and short tandem repeat profiles of these cell lines were confirmed. Both cell lines are highly aggressive and can form xenograft tumors in athymic nude mice. The MIA PaCa-2 cell line is poly-morphic, and two distinct morphological patterns are present in this cell line. In contrast, the Panc-1 cell line is pleomorphic. MIA PaCA-2 is CD24⁽⁻⁾, CD44⁽⁺⁺⁺⁾, and CD133/1⁽⁻⁾. Panc-1, on the other hand, is CD24^{(-/+)}, CD44⁽⁺⁾, CD326^{(-/+)}, and CD133/1⁽⁻⁾. Both cell lines have K-Ras and TP53 mutations, homozygous deletions including the first three exons of CDKN2A/p16INK4A, and CYR61/CCN1-overexpression. However, no mi-crosatellite instability exists. The mycoplasma for cell culture was tested in our laboratory using Mycoplasma Detection kits according to the manufacturer's instruction every three months. These cells were grown and maintained as described previously. Briefly, MIA PaCa-2 cells were cultured in DMEM (Dulbecco's modified Eagle medium), and Panc-1 cells were grown in high-glucose DMEM (Thermo Fisher Scientific) containing 10% fetal bovine serum (FBS) and 1% penicillin/streptomycin solution (Hyclone, Logan, Utah, USA). Cells with four to six passages were used for each experiment. The cells were subcultured using 0.25% trypsin/1 mM EDTA solution (Thermo Fisher Scientific) when they reached approximately 70% confluency. All cells were cultured at 37° C. in a 95% air and 5% CO₂ humidified incubator (Sanyo).

Chemical Characterization of Synthesized Polymers

To validate the chemical structures of synthesized polymers, ¹H NMR spectra was recorded by a Bruker 400 MHz spectrometer using TMS as the internal standard. Infrared (IR) spectra were recorded using an ATR diamond tip using a Thermo Scientific Nicolet 8700 FTIR instrument. To estimate the molecular weight, gel permeation chromatography (GPC) of synthesized polymers was performed on a GPC system (EcoSEC HLC-8320GPC, Tosoh Bioscience, Japan) using a differential refractive index (RI) detector as described earlier. Polystyrene (Agilent EasiVial PS-H 4 mL) was used as a standard. The sample concentration used was 1 mg/mL, of which 20 μL was injected. Dynamic light scattering (DLS) measurements were carried out using a Malvern Instrument (Malvern ZS 90). UV-visible and fluorescence spectra were recorded using a Varian UV/Vis spectrophotometer and a FluoroLog 3 fluorescence spectrophotometer, respectively. TEM studies were carried out using a JEOL JEM-2100 LaB6 transmission electron microscope (JEOL USA, Peabody, Mass.) with an accelerating voltage of 200 kV. Atomic Force Microscopy (AFM) studies were carried out according to the protocol in ACS Biomater. Sci. Eng. 2019, 5, 1354-1365.

Synthesis of pH-Responsive Block Polymers

PEG-b-poly(carbonate) was the major polymer precursor that was synthesized according to a previously described method using PEG (Mn=5000 g/mol). Ring-opening polymerization of penta fluorophenol-protected bis(methoxy propionic acid) derivative was prepared according to the synthetic strategy proposed by Hedricks et al. in J. Am. Chem. Soc. 2010, 132, 14724-14726 and later modified by Chilkoti et al. in Angew. Chem., Int. Ed. 2015, 54, 1002-1006. The resulting macromolecular precursor, PEG-b-poly(carbonate), was post-functionalized stoichiometrically using N,N′-dibutylethylenediamine (pKa=4.0) to yield the pH-responsive block copolymer PEG-DB [PEG-b-poly-(carbonate) appended with N,N′-dibutylethylenediamine side chains]. Another polymer was synthesized wherein the N-hexylamine side chain was attached to the PEG-b-poly(carbonate) precursor to form block copolymer poly(ethylene)glycol hexylamine (PEG-HX). This polymer was used throughout the study as a control amphiphilic copolymer with no pH-responsive properties. The macromolecular products were characterized by ¹H NMR and IR spectroscopy and their acid dissociation constant, i.e., pKₐ values were evaluated.

Preparation of ERKi and GEM-Loaded Nanoparticles from the pH-Responsive Block Copolymer

The self-assembled structures of amphiphilic block copolymers PEG-DB or PEG-HX in the form of nanoparticles bearing ERKi and GEM were prepared by a nonsolvent-induced phase separation (nanoprecipitation) method using DMSO as the selective solvent and PBS (pH 7.4) as the nonselective solvent. To form nanoparticles, 10 mg of PEG-DB or PEG-HX and ERKi or GEM were dissolved in 250 μL of DMSO. The resulting solution was added dropwise to 750 μL of PBS buffer (pH 7.4), and the resultant solution was transferred to a float-a-lyzer (MWCO=3.5-5 kDa) to dialyze against ˜700 mL of PBS buffer (pH 7.4) overnight. The dialyzed suspension was passed through a 0.2 μm PES filter to remove unencapsulated drugs to obtain PEG-DB or PEG-HX nanoparticles (NPs). The particle size of the filtered, drug-loaded nanoparticle suspension was measured using dynamic light scattering (DLS) studies at a scattering angle of 90°. The surface charge (C potential) of the carriers was measured either in the presence or in the absence of 10% fetal bovine serum (FBS) to determine the nanoparticles' plasma stability as a function of time. To identify in vivo stability and the fate of the nanoparticles, Alexa Fluor 647 was encapsulated within the block copolymeric nanoparticles using the nanoprecipitation method. The release rate of the encapsulated dye was monitored as a function of time.

In Vitro Drug Release Experiments

To identify the kinetics of ERKi release from pH-responsive drug carriers, the protocol reported in ACS Biomater. Sci. Eng. 2019, 5, 1354-1365 was followed. Briefly, 1 mL of the drug-loaded nanoparticle solution in a float-a-lyzer (MWCO 3.5-5 kDa) was taken. The carrier solution was then dialyzed against 5 mL of buffer solution of the desired pH either in the presence or in the absence of 10% FBS. The amount of drug released from the carrier system was measured in the bulk phase by withdrawing a specified volume of the solution from this phase periodically and replacing it with an equal amount of fresh media to maintain the sink condition.

Cell Viability Assay

PDAC cells (5000 cells/well) were seeded in 96-well plates and, 24 h later, cells were treated with different concentrations of nanocapsulated SCH 772984, DEM, or their combinations. Control cells were treated with an equivalent level of DMSO. Drug concentrations were optimized for the cell-based assay of the individual drug. At 72 h after treatment, the cell viability assay was performed by adding 10 μL of the 3-(4,5-dimethylthiazol-2-yl)-2,5-diphenyltetrazolium bromide (MTT) reagent to each well, and the plates were incubated for 3 h at 37° C. The MTT reagent was removed, DMSO (100 μL/well) was added to solubilize the crystals, and absorbance was measured at 570 nm. Synergy analysis was performed using CompuSyn software.

Clonogenic Survival Assay

PDAC cells were seeded at a density of 400 cells/well in 6-well plates. The following day, cells were treated with nanoencapsulated ERKi at a concentration of 0.01 nM, carrier-encapsulated GEM at a concentration of 0.1 nM, and a combination of 0.01 nM encapsulated ERKi, and 0.1 nM encapsulated GEM for 12 days. After the incubation time, the medium was removed, and the cells were fixed with 3:1 methanol/glacial acetic acid for 15 min at room temperature. After the fixing reagent was removed, the cells were stained with 0.5% crystal violet solution (in methanol) for 15 min. The plates were washed thoroughly with tap water to remove residual crystal violet. The plates were dried overnight, and the colonies were counted using Colony Doc-It imaging station using Colony Doc-It imaging software.

Western Blotting

Western blot analysis was the same as described in Mol. Cancer Ther. 2019, 18, 788-800. Briefly, cells were seeded at a cell density of 500,000 cells/well in 6-well plates. About 60% of confluent cells were treated with nanoparticles alone (NPA), free ERKi (FEI), or encapsulated ERKi (EEI) for 3 h at 0.001, 0.01, 0.1, 1, and 10 nM concentrations of the drug. Cells were lysed in a lysis buffer (50 mmol/L Tris pH7.4, 150 mmol/L sodium chloride, 1% NP-40, 0.25% sodium deoxycholate, 1 mmol/L EDTA) containing PMSF and Protease/Phosphatase Inhibitor Cocktail (Cell Signaling Technologies). Proteins were quantified with the Pierce BCA protein assay kits (Thermo Fisher Scientific). Under reducing conditions, a 40 g sample was loaded onto 10% SDS gels for the separation of proteins, and the resulting gel was transferred onto a 0.2 m nitrocellulose membrane. The blots were then immersed in a 5% blocking buffer made in Tris base and Tween-20 followed by incubation with antibodies against total-ERK and phospho-ERK primary antibodies (Cell Signaling Technologies; diluted in blocking buffer at 1:1000) overnight. The secondary antibody was incubated for 1 h at room temperature. The signal from the blots was detected using the Super Signal West Femto Maximum Sensitivity substrate (Thermo Fisher).

Confocal Microscopy

Cellular uptake of nanoparticles constituted from PEG-b-poly(carbonate)-derived block copolymers was assessed by confocal fluorescence microscopy. The confocal images were taken using a Zeiss Axio Observer Z1 microscope equipped with an LSM700 laser scanning module (Zeiss, Thornwood, N.Y.) at 40× magnification with a 40×/1.3 Plan-Apochromat lens. MIA PaCa-2 cells were seeded in ibidi glass-bottom dishes (35 mm) at 1×105 cells per well and grown overnight. Cells were incubated with fluorescent label (10 mg/mL, Alexa Fluor 647) at 37° C. in a high-glucose DMEM medium for 24 and 48 h. At the end of this period, cells were washed with PBS, followed by the addition of Opti-MEM, and then imaged using a Nikon Eclipse 90i Microscope. The fluorescence intensity was quantified using NIS Elements BR Software and verified by the ImageJ 1.45s Software (NIH, Bethesda, Md.).

Immunofluorescence (IF) and Immunohistochemical (INC) Analyses of Tumor Xenograft Tissues

The IF and IHC were conducted as described in Mol. Cancer. Ther. 2013, 12, 25S and Biol. Chem. 2018, 293, 4334. Briefly, five-micron-thick sections were immunostained with antibodies to Ki-67 (Santa Cruz Biotechnology) or phospho-ERK1/2 (Santa Cruz BioTechnology). Ki67 was detected immunofluorescently following counterstaining with DAPI. ERK was revealed using IHC Kits from Vector Laboratories, followed by DAB, and subsequently counterstained with hematoxylin.

Images of tissue sections stained by IF or IHC were taken using a Nikon Eclipse 90i Microscope. The number of labeled cells and stained intensity was estimated using NIS Elements BR software attached to the photomicroscope system. At least three tumors from three different mice and five sections from each tumor were used for these studies. All photographs were organized using Adobe Photoshop 2020.

Liquid Chromatography with Mass Spectrometry (LC-MS) Analysis

LC-MS analysis of drugs was performed on a Q-TOF Synapt G2S mass spectrometer (Waters, Milford, Mass.) coupled with an Acquity UPLC system (Waters). Drugs were resolved on an ACUITY UPLC HSS T3 column (1.8 M, 100 Å pore diameter, 2.1 mm×150 mm, Waters) with an acetonitrile/water gradient containing 0.1% formic acid. GEM was detected as [M+H]+, and SCH 772984 was detected as [M+2H]2+ in a positive ESI mode.

Mouse Xenograft Tumor Studies

Eight-weeks old female/male athymic nude (Foxn1nu/Foxn1nu) mice were injected subcutaneously with 0.1 mL of 2×106 MIA PaCa-2 Cells with a Matrigel basement membrane matrix (mixed at 1:1 ratio) into the hind flank. The mice were randomized (n=4-7/group), and the treatment started when the tumor volume reached 50 mm3 at 11-13 days after injection. The mice were assigned into three groups: untreated (PBS control), a combination of free SCH772984 (75 mg/kg) and GEM (40 mg/kg), or a combination of pH-responsive nanocapsule SCH772984 (75 mg/kg) and GEM (40 mg/kg). The drugs were intraperitoneally injected twice per week for 8 weeks. The tumor growth was measured according to Mol. Cancer. Ther. 2019, 18, 788-800. Briefly, tumor volume was measured using a digital caliper every 4 days, and average tumor volumes were calculated using the following formula: V=(L×W2)/2, where V=volume, L=length (the largest length), and W=width (the shortest length). After the treatment period, tumors were harvested and processed by the NDSU Advanced Imaging and Microscopy Laboratory for KI-67 immunohistochemistry for cell proliferation studies.

Tumor xenograft work complied with NIH guidelines, and all experiments were carried out following the local ethical committee (approval no.: A-19008, the drug delivery platform for pancreatic cancer using nanoparticles). IACUC was regularly monitored by inspections under North Dakota State University laws. The number of mice was considered based on power analysis, as described previously.51 The criteria of humane end points of tumors of a mouse (˜25 g) were 1. Tumor burden was greater than 15% of the body weight, and mean tumor diameter ≥20 mm, which is less than 4000 mm3 or 4 g.

Statistical Analysis

Data were analyzed by SPSS 17.0 and GraphPad Prism 8.0 software using a two-sided Student's paired t-test for single comparisons and one-way ANOVA with LSD posthoc test for multiple comparisons. Quantitative data were presented as mean±SD. Moreover, Bonferroni's correction was used to adjust for multiple comparisons. Statistical significance was at P<0.05.

Synthesis of pH/Hypoxia Responsive Diblock Copolymers

Polymers were synthesized by previously described methods. Briefly, pentafluorophenol and bis (MPA) was used to generate polymer precursor PEG-b-poly(carbonates) via a Ring opening polymerization reaction. Furthermore, the PEG-b-poly(carbonates) was modified with 2-pyrrolidin-1-yl-ethyl-amine (pKₐ=5.4) to render the copolymer pH/hypoxia-responsive in nature. The synthesized polymers were primarily characterized by I.R. spectrometry and ¹H NMR spectroscopy.

Nanoparticle Preparation

The hydrophobic drug ERKi was encapsulated with pH responsive block copolymer synthesized by the above-mentioned strategy using non-solvent induced phase separation or nanoprecipitation method. The copolymer and ERKi was dissolved in DMSO and added dropwise in PBS (pH-7.4) under stirring condition. The nanoparticles (termed as PEG-PY systems) were further dialyzed using a float-a-layer followed by filtration using 0.45μ PES filter to eliminate unencapsulated drugs. The filtered nanoparticle suspension was characterized by measuring the particle size and surface charge ((ζ-potential) using dynamic light scattering (DLS) instrument. To prepare the dye conjugated nanoparticles, Alexa Fluor-647 was co-encapsulated within the block copolymer during nanoprecipitation method.

Preparation of iRGD Conjugated Nanoparticulate Formulation

The iRGD peptide was conjugated with pH responsive block copolymer using “click” chemistry. 32 μl of sodium ascorbate and 32 μl of Cu-ascorbate complex ((27 mg/mL in deionized water) for 10 mg of polymer was added under stirring condition and kept overnight at room temperature. Following that, the materials were transferred to a dialysis bag (MWCO 1000 Da) with change of dialysis media carried out every 24 h for 72 hrs. The dialysis product was freeze-dried and then analyzed.

Drug Release Study of ERKi Nanoparticles

The drug (ERKi) released from pH/hypoxia responsive nanoparticles was measured using a float-a-layer (MWCO 3.5-5 kDa). Nanoformulation suspended in 1 mL of PBS was placed inside the float-a-layer and was dialyzed against 5 ml of PBS buffer either in the presence of or absent of 10% lysis buffer. The drug amount released in different time point was measured by the withdrawal of 1 ml dialysis media and replacement of the same volume of fresh media to maintain the overall constant volume of the system.

Cell Culture Maintenance

The two pancreatic cancer cell lines, MIA PaCa-2 and PANC-1 were procured from American Type Culture Collection (ATCC). Both cell types were cultured in high DMEM glucose Medium (Thermo Fisher Scientific) with 10% fetal bovine Serum (FBS) and 1% v/v Penicillin-Streptomycin (pen-strep). The cell lines were sub-cultured using enzymatic digestion of 0.25% trypsin/1 mM (Thermo Fisher Scientific) upon approximately reaching 70 percent confluency.

Cellular Viability Assay

Two pancreatic cancer cell lines MIA PaCa-2 (5,000 cells/well) and Panc-1 (5,000 cells/well) were seeded in 96-well plates and after 24 h, the cells were treated with the particular concentrations of SCH 772984 (0-100 nM), Gemcitabine (0-100 nM) and their combinatorial doses. ERKi was administered either in unencapsulated or in nanoparticulated form. Cells were treated for 72 h, after which cell viability was evaluated by adding 10 μL of 3-(4,5-dimethylthiazol-2-yl)-2,5-diphenyltetrazolium bromide (MTT) reagent in each well to check the cell viability followed by incubating plates for 4 h at 37° C. The absorbance was measured at 570 nm. Synergy analysis was performed using COMBENEFIT software.

Cellular Uptake Studies

The MIA PaCa-2 cells were plated at 5000 cells/well in Ibidi® glass-bottom dishes (35 mm) at 1×10⁵ cells and grew to about 70 percent confluency. Then, the cells were incubated with dye (Alexa Fluor 647) conjugated nanoparticles. The cells were washed with PBS followed by imaging using a Zeiss AxioObserver Z1 microscope equipped with an LSM700 laser scanning module (Zeiss, Thornwood, N.Y.), at 40× magnification with a 40×/1.3 Plan-Apochromat lens. For flow cytometric analysis, the cells were seeded in six well cell culture plate and after trypsinization, suspended in PBS buffer. The percentage of labelled nanoparticle uptake by the cancer cells was analyzed using BD Accuri C6 Flow Biosciences Cytometer. All the experiments were conducted in triplicate sets.

Wound Healing Assay

The MIA PaCa-2 cells were plated (10′ cells) into each well of the 6 well cell culture plate. Cells were incubated for one day at 37° C. and 5% CO₂ condition to get a confluent monolayered cells. A scratch was made in each well of the plate and images were captured. Previous cell culture media was replaced by the fresh medium before drug treatment as well as control wells. The six well plates were incubated inside the cell culture incubator for 72 h. After completion of 72 h drug treatment time, bright field images of all the wells were captured. We investigated the difference of the gap made by the scratch in all the wells (Treatment and control wells) before and after treatment. Post 24 h, cells were fixed using 4% paraformaldehyde and stained with Phalloidin. Wound area was observed under the microscope, using fluorescein isothiocyanate (FITC) filter and 10× objective lens.

Clonogenic Survival Assay

MIA PaCa-2 cells were seeded in 6-well plates with a density of 400 cells/well. Cells were kept overnight and later were incubated with ERKi at a concentration of 10 pM, unencapsulated GEM at a concentration of 100 pM, and with a combination of 10 pM encapsulated ERKi and 100 pM unencapsulated GEM for 10 days. After the period of incubation, the media was discarded, and cells were fixed with 3 part of methanol and one part of glacial acetic acid at room temperature for 20 minutes. Then, the reagent was discarded, and 0.5% crystal violet solution in methanol was applied for staining for approximately 20 minutes. Tap water was used to wash the plates to remove residual crystal violet and the samples were dried overnight. The colonies formed by PDAC cells treated with different chemotherapy formulations were then counted manually.

Identification of Cellular Death Induction Mechanism

The cell death mechanism induced in pancreatic cancer cell, i.e. MIA PaCa-2 was measured by MitoProbe JC-1 assay kit (Thermo Fisher Scientific, USA) using flow cytometry to determine the changes in mitochondrial membrane potential as a result of apoptosis. The cells were treated with the IC₅₀ concentration of GEM and ERKi nanoparticles and incubated for 24 h, 48 h and 72 h and mitochondrial depolarization was determined by the measurement of associated fluorescence using a BD-Acquire flow cytometer.

2. Characterization of pH-Responsive Nanoparticle (pH NP) System

Like other solid cancers, hypoxic niches (e.g., pO₂ less than 10 mm of Hg) of PDAC are mostly located more than 50 nm inside the surface of the tumor. The hypoxic microenvironment of solid tumors is directly linked to a pH drop from 7.4 (normoxic) to <6.6, associated with aggressive and metastatic phenotypes. In addition to hypoxia, enhanced glucose uptake and carbonic anhydrase activity on the cancer cell surface also contribute to creating an acidic extracellular milieu favoring tumor growth, invasion, and the emergence of GEM-resistant metastatic phenotypes of PDAC. Given the importance and exploiting the acidic/hypoxic environment in PDAC, pH-mediated activation of the carrier system for drug deliveries is an ideal option to increase the intratumor concentration and limit off-target distribution of drugs as previously shown. The carrier system was also designed to harness the local pH and hypoxic conditions of PDAC for activating drug release.

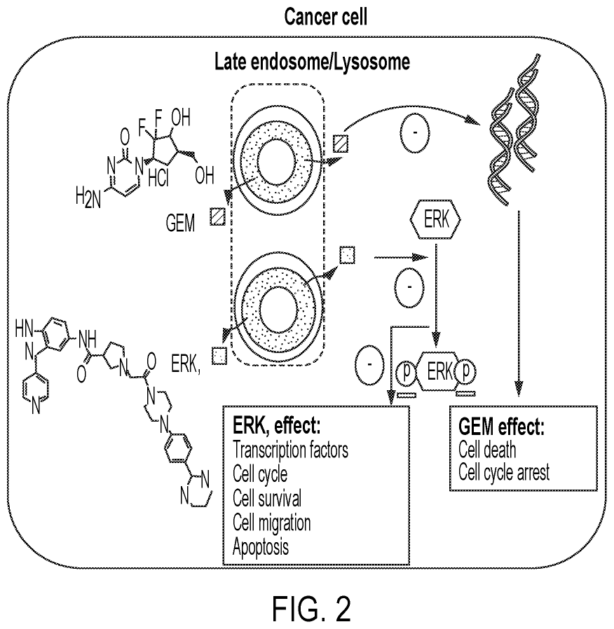

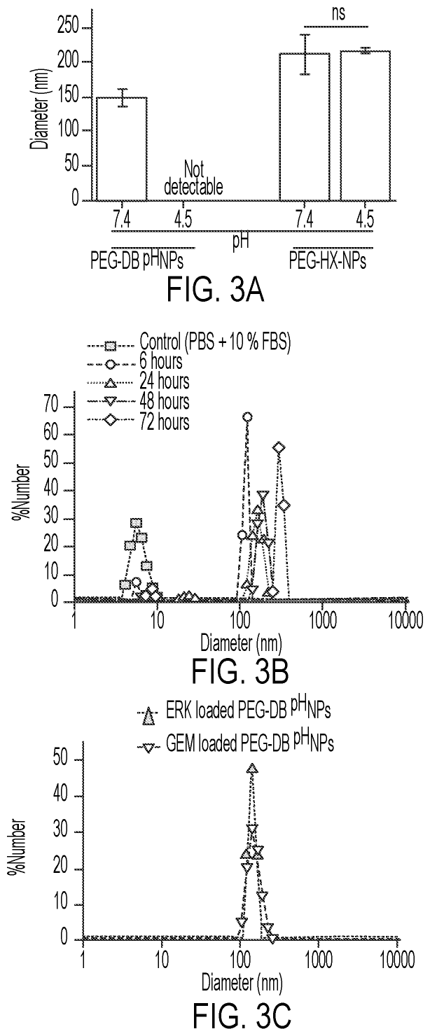

A pH-dependent NMR investigation on polymer PEG-DB was first performed. It was found that, at the low pH (i.e., 5-4.5) of the culture media, the side chain amines were protonated, thereby leading to peak broadening of signals of —CH₂— units associated with tertiary nitrogen. A PEG-b-poly-(carbonate)-derived block copolymer was synthesized, where N,N′-dibutyl ethylene diamine side chains were appended at the poly-(carbonate) segment as shown in FIG. 2. This copolymer exhibited a pKₐ value of 5.53±0.14, thus showing a pH-dependent assembly-disassembly behavior. The pKₐ was determined by estimating the pH at the half-equivalence (inflection) point of the titration curve, where at least 50% of the amine groups located in the block copolymers are in the protonated form. This protonation promotes the amphiphilic to the hydrophilic transition of the macromolecule, leading to destabilization of their self-assembled structures that would otherwise be stable at a plasma pH of 7.4. It was also reported that the critical aggregation concentration (CAC) of copolymer 4 was 8.5×10⁻⁶ M at pH 7.4, which is comparable to polyesters and polypeptide-derived block copolymers, indicating adequate copolymer stability at pH 7.4 for systemic administration. The experiments showed that, upon nanoprecipitation, PEG-DB block copolymers may be formed from nanoparticles within the size range of 149±14.4 nm. The size reduction of the PEG-DB block copolymeric nanoparticles was observed to be pH-dependent, while the pH-dependent reduction of particle size is not observable in PEG-HX systems as observed by DLS, AFM, and TEM based studies. The DLS data is shown in FIG. 3A.

An increment of particle size for these block copolymeric assemblies in the protein-rich solution of FBS was found as shown in FIG. 3B, which is most likely due to the deposition of protein corona. The ζ potential of drug-free nanoparticles composed of PEG-DB was found to be −2.8 mV at pH 7.4. A neutral charge on nanoparticles composed of PEG-HX systems was observed, indicating that PEG-DB particles were stabilized through steric stabilization. Drug carriers composed of PEG-DB systems were also stable in the presence of plasma for at least 72 h as shown in FIG. 3C.

3. Drug Encapsulation in a pH-Responsive Nanoparticle (pH NP)

Having characterized the pH NPs, the drug encapsulation and drug delivery capacity of pH NPs was determined. First, SCH 772984 was encapsulated into the block copolymer by nonsolvent-induced phase separation or a nanoprecipitation technique, where both the drug and the polymer were codissolved in DMSO and gradually added to water, which is a nonsolvent for both the species. The unencapsulated drug was removed by filtration and dialysis. It was found that nanoparticles, constructed from block copolymer 4, could encapsulate the drug at 12.9% loading content. The results were calculated using the following equation (n=5 statistical replicate) at 60% loading efficiency:

$$loading content = \frac{total mass of drug loaded}{total weight of the formulation} \times 100$$

No precipitation of ERKi was observed from this colloidal suspension, which indicated the formulation stability of these systems. The same procedure for encapsulating GEM into the block copolymer was used, in which the drug can be encapsulated at 12.9% loading content. Earlier, it was reported that the encapsulation efficiency of PEG-DB nanoparticles for GEM was 86±3.18%, and the loading content was 29±7.5%. Dynamic light scattering (DLS) studies revealed that the diameter of ERKi-loaded nanoparticles was 142.8±20.9 nm, while GEM-bearing nanoparticles exhibited a diameter of 144.8±33.4 nm in PBS (FIG. 2D). Transmission electron microscopic analysis also revealed the formation of nanoparticles in the form of particles of uniform size.

Since the ratio of the mass of the hydrophobic block to the total polymer mass (known as the f value) of the block copolymer is maintained between 0.35 and 0.45, the constructs are most likely to have polymersome-like architectures where the ERKi molecule SCH 772984 could be localized within the bilayer structure of the polymersome membrane. Assembly of polymersomes is mostly driven by the hydrophobic interactions among the nonpolar domain of the constituent block copolymer and stabilized by PEG shell. It was envisioned that enhanced hydrophobic interactions of the polycarbonate domains of PEG-b-poly-(carbonate) block copolymers and the pH-specific protonation capacity of the tertiary amines located at poly(carbonate) segment would generate a systematically stable, spatiotemporally controlled ERKi release from the nanoparticle only within the acidic compartments of PDAC.

4. pH-Dependent Drug Release from Nanoparticles

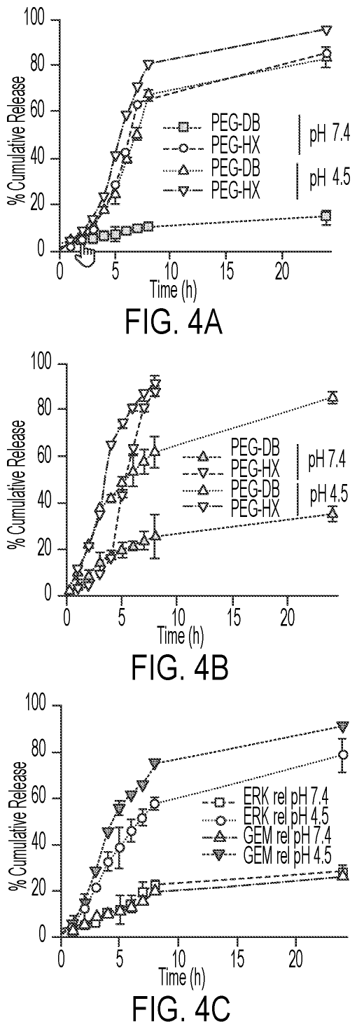

To evaluate the therapeutic potency of drug-encapsulated pH NPs, the drug-release capacity of ERKi-pH NPs or GEM-pH NPs in the media of a different pH was first determined. It was assumed that these pH NPs initiated drug release under the influence of pH in the intratumoral space of the PDAC microenvironment or intracellular acidic compartments such as endosomal-lysosomal pathways (pH 5.5-4.5). The drug concentrations in the media were measured with a different pH (7.2-4.5), following treatment with drug-loaded pH NPs. The measurement was performed for 24 h at a regular interval and the results are shown in FIG. 4A and FIG. 4B. The release media in all cases were spiked with 10% FBS to identify the effect of plasma proteins on drug release. The cumulative percentage release of ERKi, SCH 772984 from PEG-DB nanoparticles was found to be >80% at the end of 24 h at pH 4.5; however, only 15.2±4.0% release was observed at pH 7.4. Yet, when ERKi was loaded in non-pH-responsive control nanoparticles, >60% of drug was released irrespective of pH within 8 h. Similar results were obtained with GEM-loaded nanoparticles; the extent of cumulative release of the drug significantly increased at pH 4.5 (85.1±2.5%) compared to pH 7.4 (35.2±3.47%) after 24 h. Both release studies indicate that pH-responsive nanoparticles efficiently controlled the drug release. This study collectively suggests that pH NPs could be activated under the influence of low pH conditions found in the PDAC microenvironment.

Whether the combination treatment of the ERKi-p^{H}NPs (pH-responsive nanoparticles encapsulating ERK inhibitor) and GEM-p^{H}NPs (pH-responsive nanoparticles encapsulating GEM) would selectively release both drugs under low pH media was then determined. To do so, a mixed nanoparticle formulation in which ERKi- and GEM-p^{H}NPs were mixed at a 1:1 volumetric ratio was prepared and subjected to release conditions in pH-adjusted, serum-containing media. As shown in FIG. 4C, it was found that an acidic pH of 4.5 activated release for both drugs (>20% in the first 7 h), but a neutral pH of 7.4 did not trigger drug release to a significant extent (<20% in the first 7 h) for both of the drugs. These results indicate that amphiphilic PEG-DB systems can be used as drug carriers for both ERKi and a combination formulation of the drug with GEM, where the maximum release of the drug(s) can be achieved only in acidic compartments but not in the systemic environment. The drugs' selective delivery will help attain significant enrichment of the drug in PDAC tissues with minimized off-target effects.

5. In Vitro Cytotoxicity and Synergism of ERKi and GEM-Encapsulated pH NPs

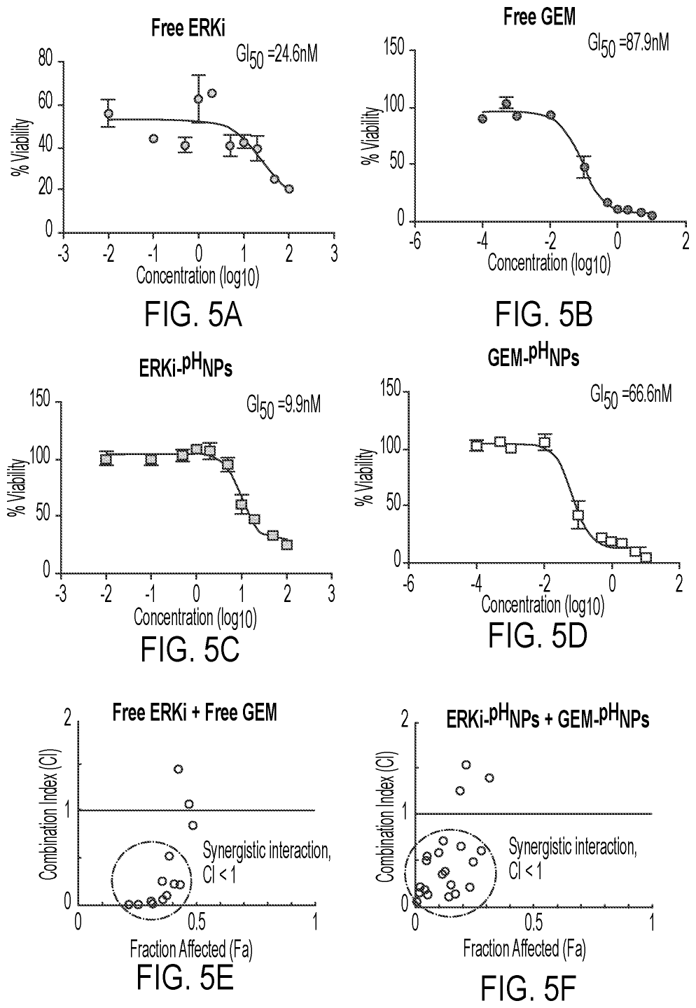

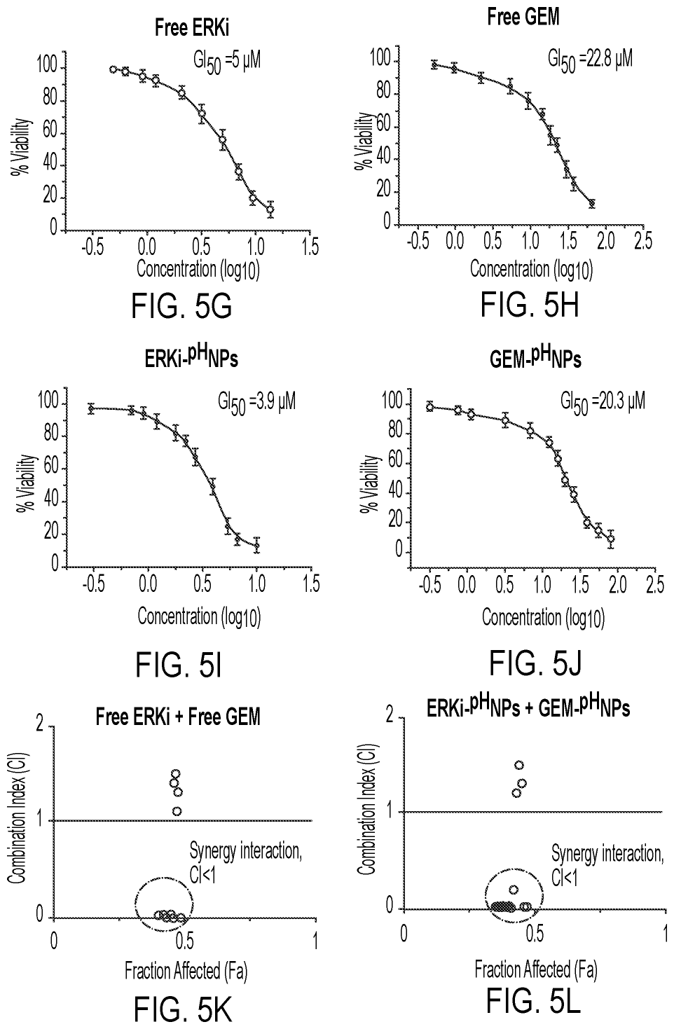

In this study, to determine whether combining ERKi, SCH 772984, with GEM exhibits a synergistic cytotoxic effect and whether such effect persists upon encapsulation of the active agents, a series of in vitro cytotoxicity studies were conducted in two different PDAC cell lines (MIA PaCa-2 and Panc-1). The data was fitted in CompuSyn. These two cell lines were selected because MIA PaCa-2 cells are responsive to ERKi and GEM compared to Panc-1. As shown in FIG. 5, it was observed that the IC₅₀ for free SCH-772984 and GEM was markedly higher than encapsulated drugs in MIA PaCa-2 and Panc-1 cell lines. However, consistent with previous studies, the Panc-1 cells were more resistant to these drugs compared to MIA PaCa-2 cells.

Furthermore, it was found that, out of 25 concentration combinations of ERKi and GEM, 13 showed synergistic activity (CI<1, FIG. 5E and FIG. 5K), and the synergism was maintained when the drug molecules were encapsulated within the amphiphilic copolymer-derived nanoparticles (FIG. 5F and FIG. 5L). ERKi- or GEM-encapsulated non-pH-responsive systems showed less efficacy than their pH-responsive counterparts. These results indicate a dose-dependent effect of drug-free NPs on cell viabilities of PDAC cells. However, low doses of free NPs, which are used in vitro therapeutic applications, showed no impact on cell viability of PDAC cells.

6. pH-Responsive Nanoparticle Uptake by the PDAC Cell Line

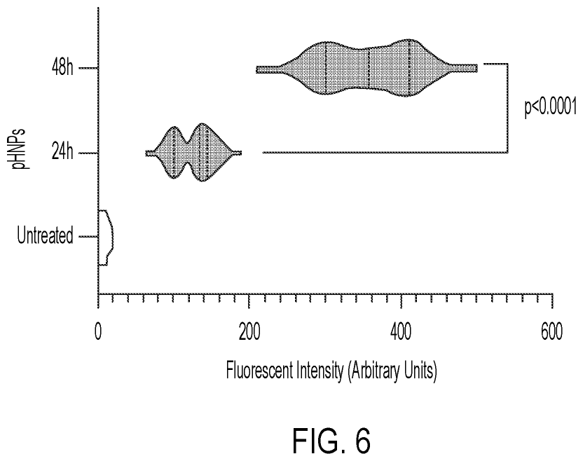

We studied a time-dependent uptake of fluorescently labeled nanoparticles by MIA PaCa-2 cells to determine the prognostic and therapeutic efficacy of pH-responsive nanocarriers (^{PH}NPs). As shown in FIG. 6, fluorescently labeled nanoparticles were internalized through the cell membrane and accumulated mostly in the cytoplasm, and the maximum intensity was detected after 48 h of incubation. The control (PBS-treated) shows no labeled cells. The results suggest that the uptake of the nanoparticles by PDAC cells is time-dependent. The time-dependent disparity in the uptake of nanoparticles has important implications for designing nanoparticle-based drug delivery systems.

7. Differential Suppression of ERK Activity in Free or ERKi pH NPS-Treated Cells

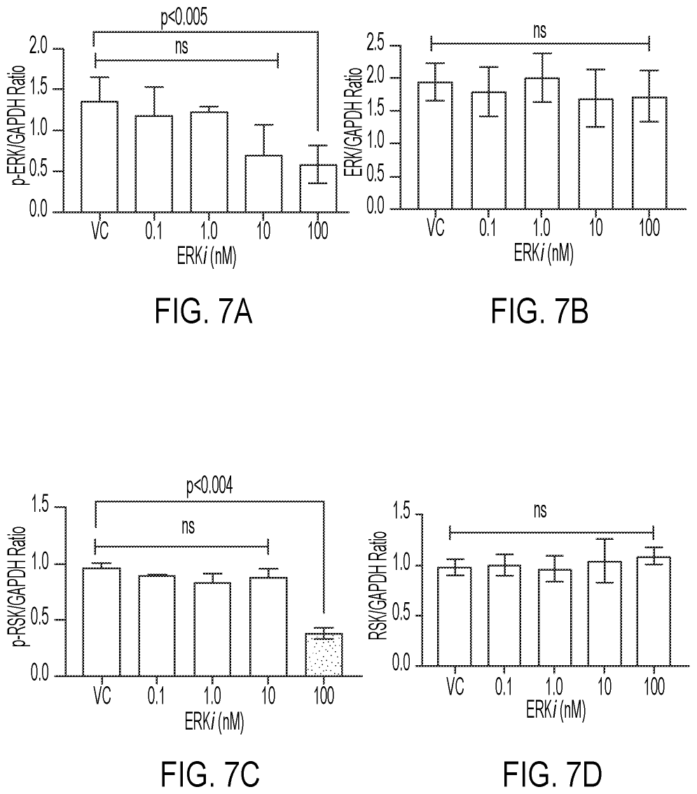

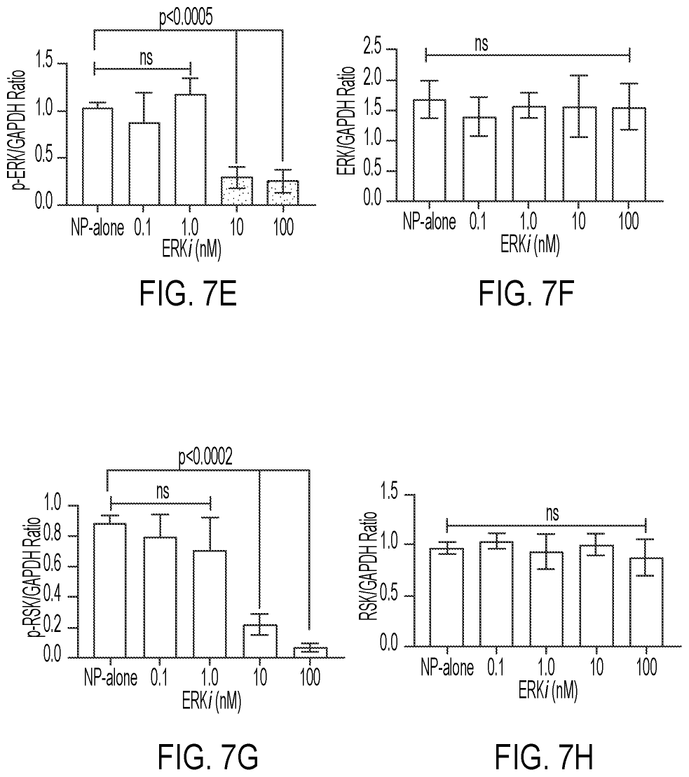

To determine the functional efficacy of ERKi-pH NPs, the question of whether encapsulated ERKi SCH 772984 can suppress the ERK activity by suppressing ERK phosphorylation (p44/p42 MAPK/ERK1/2) in PDAC cells was investigated. To do so, MIA PaCa-2 cells were exposed to nanoparticles free of SCH 772984 or encapsulated with SCH 772984 with different doses (0.1-100 nM) for 3 hours. Following treatment, total, phospho-ERK, RSK, and GAPDH were determined using Western blot analysis in the cell extracts. SCH 772984 (free or encapsulated) treatment did not affect total ERK (ERK) and total RSK expression in MIA PaCa-2 cells. However, p-ERK and p-RSK levels, indicators of functional activity, were markedly decreased by free ERKi and encapsulated ERKi in a dose-dependent manner as shown in FIG. 7. The inhibitory effect of free ERKi was first detected at a concentration of 100 nM. In contrast, the inhibitory effect of encapsulated ERKi was first identified at a concentration of 10 nM in MIA PaCa-2 cells. A nonsignificant change of the pERK level was detected in free ERKi at a dose of 10 nM, which was 2.4-fold less than 10 nM encapsulated ERKi. Collectively, this study demonstrates that ERKi-pH NPs was more effective than free ERKi.

8. ERK Promotes GEM Resistance in PDAC Cells

To examine the link between ERK and GEM resistance in PDAC cells, a Clonogenic assay was performed for anchorage-dependent growth in MIA PaCa-2 and Panc-1 cell lines in the presence or absence of free or encapsulated ERKi (10 nM) or GEM (100 nM) alone or a combination of these two drugs for 12 days. The half-effective doses of encapsulated ERKi or encapsulated GEM was purposely selected from the preceding studies shown in FIG. 6 and FIG. 7 because these doses of free-drugs have minimal or no effect on PDAC cell viability (FIG. 6) and ERK phosphorylation (FIG. 7). Moreover, the MIA PaCa-2 and Panc-1 cell lines were considered for this study because previous studies found that MIA PaCa-2 cells were sensitive to SCH772984 in vitro compared to Panc-1. As expected, ERKi-pH NPs alone has no significant impact on the survival and colony-forming ability of MIA-PaCa-2 cells. At the same time, GEM with a low dose was able to decrease the colony-forming ability of these cells significantly as compared to untreated cells.

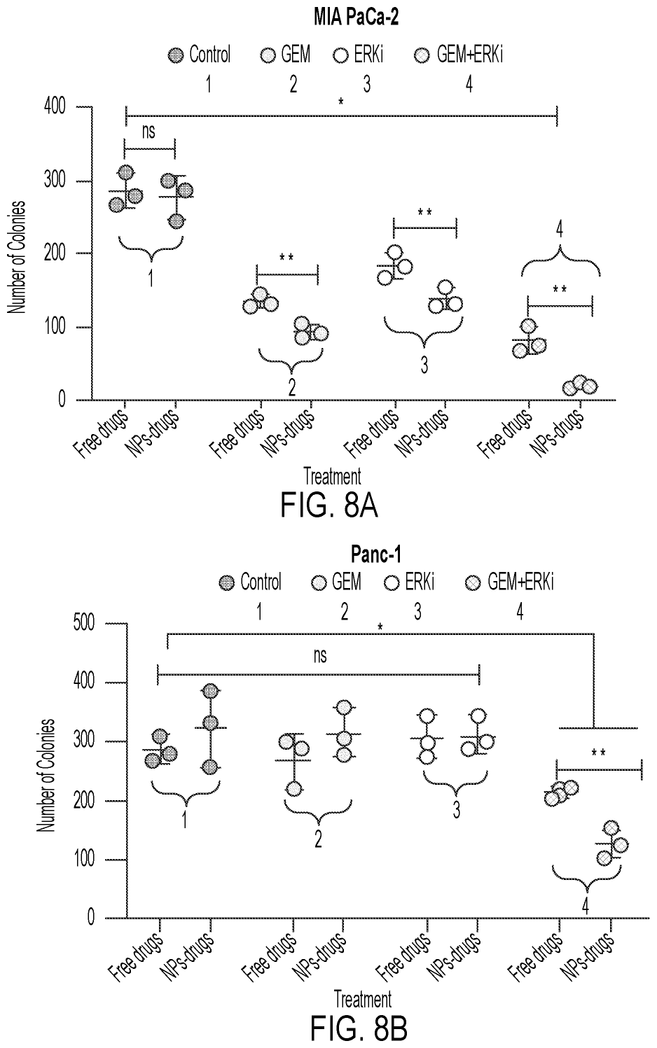

As shown in FIG. 8, it was observed that the combination treatment of ERKI-pH NPs and GEM-pH NPs significantly blocks the colony-forming ability of MIA PaCa-2 cells compared to untreated or GEM-pH NPs alone treated cells. Collectively, these findings demonstrate that ERK signaling could be responsible for promoting GEM-resistant phenotypes in PDAC cells.

9. Detection of Drug Sensitivity in the MIA PaCA-2-Tumor Xenograft Model

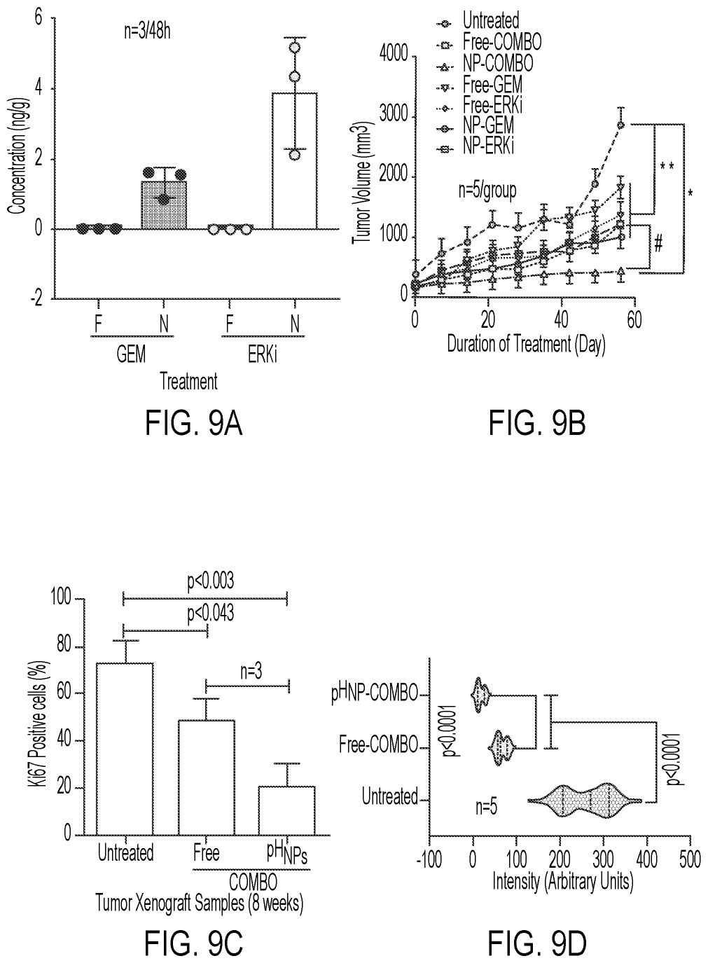

Next, in vivo studies to obtain information on the systemic stability and tumor enrichment capacity of pH NPs were performed. The fate of drug-encapsulated nanoparticles in the MIA PaCa-2-pancreatic tumor xenograft model in immune-compromised nude mice was studied. Upon intraperitoneal (ip) administration of free or drug-pH NPs [75 mg/kg for SCH772984 and 40 mg/kg for GEM; human equivalent dose (HED)] for 48 h in tumor-bearing mice (N=3), the drug concentrations in tumor tissues was measured using LC-MS. As shown in FIG. 9A, ERKi (˜2 ng/g) and GEM (˜4 ng/g) was detected in tumor tissues injected with NPs, which is typical for the ip administration of nanoparticles. The concentrations of free drugs are significantly low compared to drugs NPs.

The impact of nanoformulated drug treatments on subcutaneous tumor growth was evaluated. pH NPs-ERKi alone, pH NPs-GEM alone, or a combination of pH NPs-ERKi with GEM-pH NPs was administered intraperitoneally twice a week with PBS into tumor-bearing mice at a dose of 75 mg/kg for SCH772984 and 40 mg/kg for GEM for 8 weeks via ip injection. The tumor growths were measured every other day. It was found that the combination of drug therapies of established tumors impaired tumor progression (free COMBO) or caused tumor regression (pH NP-COMBO) (FIG. 9B) through suppressing cell proliferation as the number of Ki67, a cell proliferation marker, positive cells significantly reduced in these tumor samples as compared to untreated samples. Although free or encapsulated drug combination treatment affects pancreatic cancer growth, pH NP-COMBO resulted in a more significant tumor growth reduction than free COMBO. Furthermore, the impact of individual drugs (free- or encapsulated) exhibited significantly less effect on tumor growth than combination therapy. Together, the results suggest that pH NP-COMBO with the doses of ERKi and GEM used for this study may be beneficial for treating PDAC patients.

The ERK status in tumor samples collected from free COMBO and pH NP-COMBO was examined. It was found that phospho-ERK levels were significantly reduced in pH NP-COMBO samples than free COMBO, indicating p^{H}NP-COMBO is superior to free COMBO to suppress ERK activity (FIG. 9D).

It was investigated whether drug-free p^{H}NPs produce a cytologic toxic effect on the liver and kidney in tumor-bearing mice. The histological studies showed that carrier-forming polymers do not trigger hepatotoxicity and renal toxicity after prolonged treatment for 45 days compared with the untreated control.

10. Analysis of Experimental Results

A pH-responsive block copolymer used to encapsulate two drugs, i.e., GEM and ERKi, was generated. Encapsulation of these drugs protects them from enzymatic degradation, lowers their off-target toxicity, and increases their circulation lifetime without compromising their chemical reactivity on cellular targets. After conducting physicochemical characterization studies, the synergistic effect of these two drugs on PDAC cells' viability was established. The studies also showed that the minimum inhibitory concentration (less than IC50) of ERKi, which has no or minimal impact on PDAC cell survival, promotes GEM sensitivity significantly in these cells. Moreover, the first report that the synergistic concentrations of the drug in vivo to tumor be toned down was provided.

PDAC is a mutant K-RAS-addicted cancer. Aberrant activation of the RAS-ERK pathway contributes to evading senescence and promoting epithelial-mesenchymal transition (EMT), cancer stemness, cellular invasion, migration, and chemoresistance. However, no drug or drug combination has yet been developed that effectively blocks the oncogenic function of K-RAS. In many instances, this could be due to the reactivation of the downstream ERK-signaling pathway, which is actively involved in the oncogenic activities of mutant K-Ras.

Although GEM is a standard drug to treat pancreatic cancer, it does not kill the mesenchymal stem cells, which leads to drug resistance. Thus, direct inhibition of ERK to block K-RAS, combined with GEM, is an attractive option for PDAC therapy. Nevertheless, nontargeted inhibition of the ERK pathway causes systemic toxicity. Targeted drug delivery inside the PDAC microenvironment is also challenging as of the hypoxic-related desmoplastic barrier. The pH/hypoxia-responsive nanoparticles based drug delivery system can overwhelm the hypoxic-related desmoplastic blockade and release the drugs by exploiting the low pH microenvironment, thereby suppressing the proliferation of PDAC cells. Given the importance of these drug delivery systems, this nanoparticle was used to deliver ERKi and GEM. Since ERKi is a hydrophobic molecule while GEM is hydrophilic, amphiphilic block copolymers were used with pH-triggered side chains to encapsulate ERKi and GEM.

Physicochemical and biological studies were performed to establish the feasibility of poly(carbonate)-derived cancer microenvironment-responsive block copolymers as drug carriers alone and for combination treatment of GEM and ERKi. A formulation strategy was formed to encapsulate these two drugs of apparently opposite polarities into block copolymeric systems with sufficient loading capacity and efficiency. It was demonstrated that encapsulation of this combination within a nanoformulation markedly increased the synergistic action on cell viability as compared to free drugs. The superior effect of nanoparticles was also established in ERKi-pH NPs-treated cells, in which ERK activity was decreased in a dose-dependent fashion, and the maximum effect was detected at the IC₅₀ dose.

The possible mechanism for the enhanced activity for nanoparticle-bound ERKi and GEM activity could be attributed to extremely poor solubility of unencapsulated ERKi and enzymatic hydrolysis of unencapsulated GEM. Encapsulation into nanocarrier systems most likely increases the intracellular concentration of these two drugs by bypassing the limitations, as mentioned above, associated with the unencapsulated (free) form of drugs. This may be attributed to the small molecular drugs' easy influx and efflux without engaging the cognate drug receptors. In contrast, drugs encapsulated in the NPs are programmed to effectively release their payload at the endosomal-lysosomal space, which causes an adequate amount of drug being delivered directly and minimal drug loss due to efflux.

In addition to the synergistic effect, an unexpected finding from the studies was achieved indicating ERKi with a minimum inhibitory concentration (0.01 nM) significantly enhances the GEM's effect on PDAC cells. Although the mechanism of GEM sensitization by ERKi is unclear, the suppression of ERK may promote a reprogramming event that shifts EMT to MET (mesenchymal-epithelial transition) in PDAC cells. The resulting epithelial cancer cells become sensitive to GEM.

The pH-responsive block copolymer assemblies of GEM and ERKi formed stable nanoparticles and were able to withstand the effect of the systemic circulation. It was also shown that the pH-responsive nanoparticles were able to transport drugs at an in vivo level. As a result, PDAC tumor growth was significantly reduced by combination therapy of the pH-responsive nanocapsule ERKi with GEM as compared to free drugs where only modest antitumor activity was observed.

It was shown that amphiphilic copolymer formulations for delivery of potent combination chemotherapy having a small-molecule inhibitor of ERK and frontline chemotherapy, GEM for enhanced suppression of cancer cells. It was also shown that pH-responsive nanoparticles could transport GEM and ERKi, SCH 772984, to their cellular targets to inhibit the K-RAS-mutant pancreatic cancer growth. This study collectively suggests that a pH-responsive nanoparticle-mediated combination treatment of the ERKi inhibitor and GEM can provide a means for PDAC elimination.

11. Morphology and Stability Study of iRGD Conjugated pH-Responsive Copolymer Encapsulated ERKi

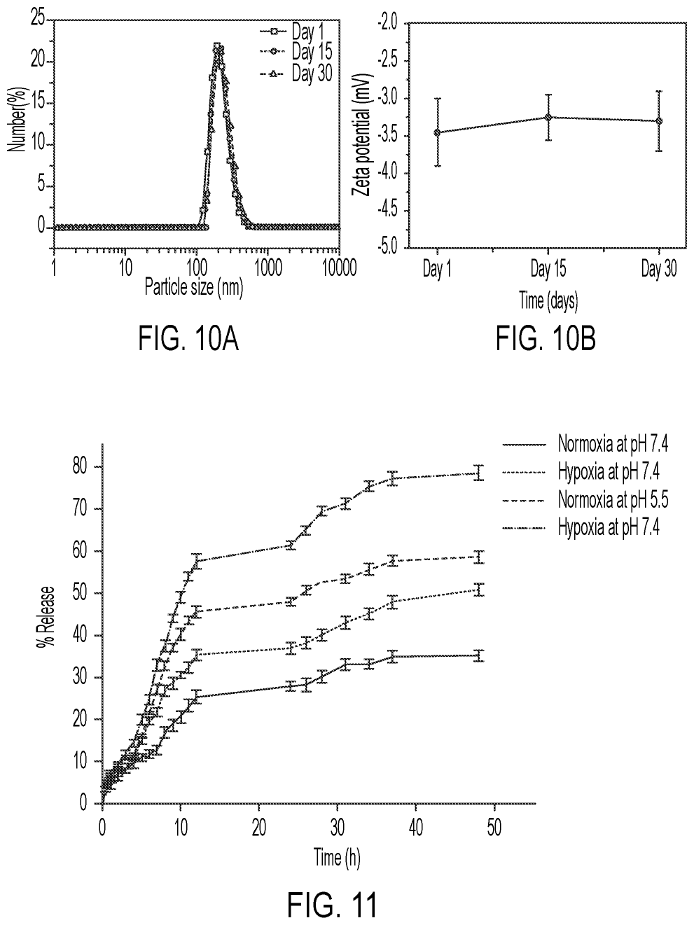

The pH-responsive nanoparticles, which were abbreviated previously as PEG-PY/pH NPs nanoparticles, are composed of poly(ethylene glycol)-b-poly(carbonate) with an N, N-pyrrolidone side chain. In addition, the surface of the PEG-PY was decorated with an iRGD peptide, a cyclic peptide composed of 9-amino acids including an Arg-Gly-Asp (RGD) motif, which acted as a PDAC homing ligand. The size, zeta potential, and self-assembly of PEG-PY were determined using transmission electron microscopy (TEM) and dynamic light scattering (DLS), as shown in FIG. 10A and FIG. 10B. Transmission electron microscopy of PEG-PY systems revealed that the pH-responsive block copolymers self-assemble to spherical nanoparticles. It was also observed that PEG-PY nanoparticles showed an average hydrodynamic diameter of 185±17 nm (FIG. 10A) with a surface charge between −3.25 to −3.45 mV, which remained stable throughout 30 days of storage periods (FIG. 10B).

12. Drug Release and Kinetic Study

In PDAC, early hypoxia triggers remodeling of the extracellular matrix acidification, epithelial-to-mesenchymal transition (EMT), cell survival, metastasis, the formation of CSCs/TICs, and significant resistance to chemo- and radiotherapy. In addition, hypoxic conditions trigger glycolytic switch in tumor cells, resulting in the cytosol and eventually extracellular matrix (ECM) acidification. Hypoxia, enhanced glucose uptake, and carbonic anhydrase activity on cancer cell surface also contribute to creating such acidic extracellular milieu favoring tumor growth, invasion, and the emergence of GEM resistant metastatic phenotypes of PDAC. Thus, drugs could also be effectively released from PEG-PY/pH NPs in hypoxic microenvironments.

A cell-free study investigated ERKi release from PEG-PY/pHNPs nanoparticles at pH 7.4, 5.5, or under hypoxic conditions (pO₂=2 mm Hg). It was found that in PBS at pH 7.4 and normoxic conditions, ˜20% cumulative release of ERKi took place within 10 hours, followed by an additional 8% cumulative drug release within the next 40 hours. However, in hypoxic conditions at pH 7.4, >40% of drug release took place after 50 hours (FIG. 11). Acidification of release media to pH 5.5 significantly increased the rate and extent of drug release. For example, it was found that under normoxic and acidic pH conditions (pH 5.5), >50% ERKi was released from nanoparticles within the first ten hours of the release experiment, which was significantly accelerated when drug release took place acidified and hypoxic conditions. Under these conditions, which are also predominantly present in the PDAC microenvironment, it was observed that more than 60% ERKi release took place after the first ten hours. In comparison, >70% of the drug was released from the nanoparticles at the end of 50 hours. As the data shows in FIG. 11, these studies collectively indicate that iRGD conjugated PEG-PY nanocarriers release the drug in low pH and hypoxic microenvironments.

13. Determination of Synergistic Interactions Between Nano-Encapsulated ERKi and Free GEM

A synergistic effect of ERKi, SCH 772984, and GEM was observed in pancreatic cancer cell lines and subcutaneous tumor xenograft model when encapsulating these drugs within PEG-PY/pH NPs nanoparticles. To conduct a synergy assay, a cytotoxicity analysis of nanoencapsulated ERKi in the presence of free GEM against PDAC cells for 72 hours was performed. COMBENEFIT software was used to analyze the data using three different synergy models such as Bliss, HSA, and Loewe to determine synergistic interactions between nanoencapsulated ERKi and free GEM. Based on the three models, nanoencapsulated ERKi was observed to exhibit a synergistic interaction with free GEM according to all three models, representing the most widely accepted surface maps of Bliss analysis.

14. Cellular Internalization of the Nanoparticles in MiaPaCa-2 Cells

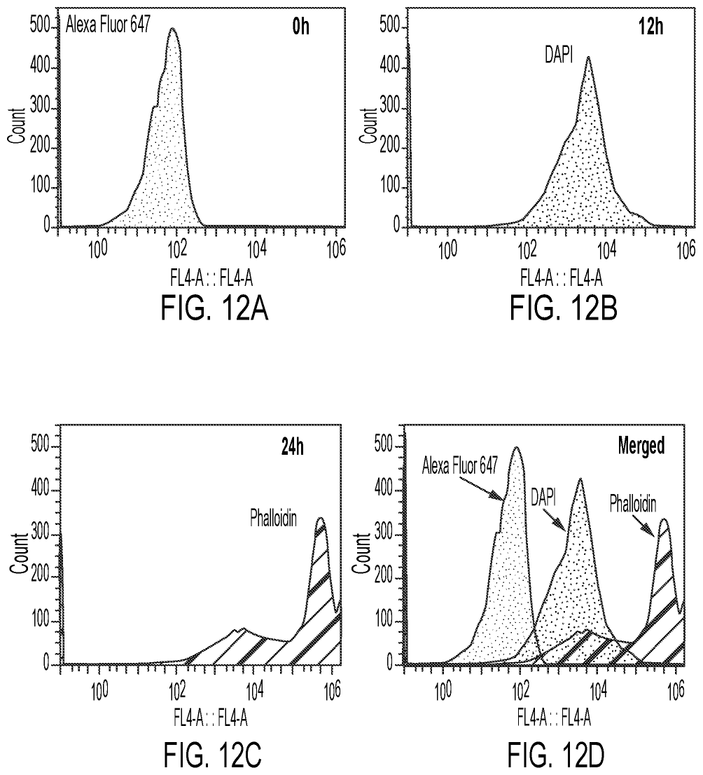

To identify the cellular internalization behavior of PEG-PY/^{pH}NPs nanoparticles, the particles were conjugated with Alexa Fluro 647 dye. Then, MIA PaCa-2 was treated with these dye-labeled nanoparticles for 12 and 24 h. A time-dependent increase of the cellular uptake of Alexa Fluor 647 labeled nanoparticles was observed. The nanoparticles were accumulated mainly in the cytosol of MIA PaCa-2 cells. Flow cytometry experiment of MIA PaCa-2 cells treated with Alexa Fluor 647 loaded nanoparticles for 12 and 24 h validated the experimental observation from confocal microscopy-based experiment, where similar time-dependent uptake of particles inside cells were observed, as shown in FIG. 12A-12D.

15. Measurement of Colony Formation Capacity after Drug Treatment

The colony formation capacity of MIA PaCa-2 cells (in a low density) was investigated post-treatment with PEG-PY/pH NPs nanoparticle encapsulated ERKi in the presence of free GEM. To conduct this experiment, cells were treated with IC₅₀-equivalent concentration of free Gem, nanoparticle-encapsulated ERKi, and the combination of both drugs (nanoparticle encapsulated ERKi in the presence of unencapsulated GEM). The significant inhibition of the colony formation has been observed compared to control for the combination treatment of nanoparticle-encapsulated ERKi in the presence of GEM. The 0.1% DMSO and empty nanoparticles treated MiaPaca2 control cells were found to form multiple colonies throughout the cell culture well. The combination treatment of free Gem and free ERKi (at IC₅₀ concentration for both drugs) demonstrated superior suppression of colony forming capacity of MIA PaCa-2 cells compared to single drug- or DMSO-treated controls.

16. Measurement of In Vitro Cell Migration after Drug Treatment

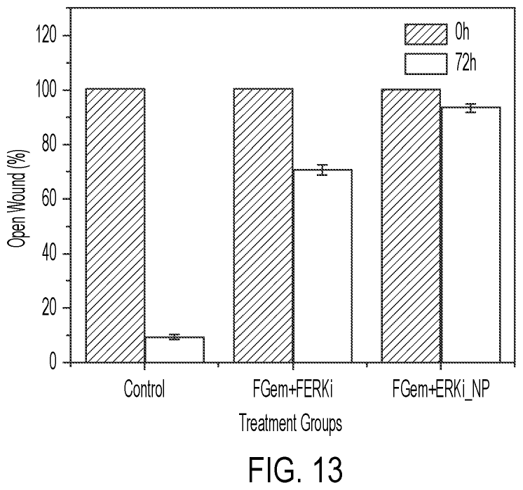

A wound healing assay was conducted to determine the ability of cancer cells to migrate post-treatment with the combination therapy (consists of nanoparticle encapsulated ERKi and GEM) compared to untreated control. These results are summarized in FIG. 13. In a confluent monolayer MIA PaCa-2, a scratch was created to remove the cells from that area of the plate. The plates were incubated with unencapsulated GEM or unencapsulated ERKi, combination of unencapsulated drugs, as well as the combination therapy consists of nanoparticle encapsulated ERKi with GEM. The effect of drug exposure on the migration rate of cancer cells was investigated. The combination of free GEM and nanoparticle encapsulated ERKi demonstrated the highest percentage of inhibition in cell migration compared to the free drug combinations. The control plate cells successfully covered the gap i.e., the cell-free area after 72 hours due to the high migration rate of cancer cells. The rate of gap closure represented as open wound percentage was calculated for different treatment groups, which indicates the free Gem and ERKi nanoparticle combination significantly prohibited the cell migration rate.

17. Evaluation of Mechanism of Cell Death

Generally, mechanism of cancer cell death triggered by chemotherapeutic drug treatment reflects the principal contribution of mitochondria in intrinsic pathway of apoptosis induction. During chemotherapeutic drug treatment, excessive ROS generation beyond a cellular tolerability threshold results disruption of the mitochondrial transmembrane potential (Δψm).

The changes in mitochondrial transmembrane potential can be quantitatively measured by cationic lipophilic dye, 5,5′,6,6′-tetrachloro-1,1′,3,3′-tetraethylbenzimidazolylcarbocyanine iodide (JC-1) using flow cytometry analysis. In normal cells, due to the highly negative membrane potential of mitochondria, the cationic dye JC-1 can easily enter inside the mitochondria and forms J-aggregates which emits red fluorescence whereas in apoptotic cells due to the depolarization of membrane potential, the dye could not enter inside the mitochondria remains at cytosol as monomeric form (emits red fluorescence). Following this principle, MIA PaCa-2 cells were stained with JC-1 dye and quantified for the extent of apoptosis induced by the drug combination after incubation of 24-72 h. The highest percentage of apoptotic cells (75.6% at 72 h, 66.1% at 48 h and 49.4% at 24 h) were observed after 72 h incubation with drug combination consists of nanoparticle encapsulated ERKi in the presence of GEM. Thus, the conversion of aggregated to monomeric form of JC-1 represented a quantitative determination of mitochondrial depolarization that directly linked to apoptosis induction in pancreatic cancer cells by the drug combination.

18. Analysis of Experimental Results

PDAC is one of the most lethal type of pancreatic cancer. Lack of early detection protocols, emergence of drug resistance, and metastasis of cancer cells to vital organs render the treatment of this disease challenging. Gemcitabine hydrochloride is the most commonly used frontline drug, used in combination with ABRAXANE and FOLFIRINOX. Almost 90% of PDAC is reported to acquire mutation in KRAS oncogene which drives the progression of the disease. Although GEM is considered as a first line chemotherapeutic drug for PDAC treatment, but it is minimally effective for long term treatment in pancreatic cancer patients. Several research findings suggest that the limitation of GEM based chemotherapy is mainly due to the drug resistance exhibited by the cancer stem cells present within the hypoxic tumor microenvironment (TME). Several signaling pathways are interconnected for the development of drug resistance properties, including the RAS mutation in MAPK signaling pathway associated with extracellular signaling regulated kinases (ERK) 1/2. The phosphorylation of MEK 1/2 activates ERK1/2 initiating principle oncogenic activities including cellular differentiation, proliferation, and motility. Several ERK1/2 inhibitors have been developed and primarily classified into groups such as ATP competitive ligands (Type I), The ERK1/2 enzymatic activity inhibitors prohibit downstream substrate phosphorylation of ERK. The promising ERK1/2 inhibitor, SCH772984 demonstrated dual activity by suppressing enzymatic activity of ERK1/2 and blocking phosphorylation of MEK. However, low water solubility and off target cytotoxicity of SCH772984 poses challenge for its clinical use. Therefore, pH/hypoxia responsive nanoparticle that encapsulated SCH772984 was developed.

It was observed that nanoparticle encapsulated SCH772984 in combination with GEM (in free, or non-encapsulated form) induced synergistic cytotoxicity by inducing apoptosis against PDAC cells, and thus could be used to delay drug resistance developed by pancreatic cancer stem cells towards the combination. The physical characterization and formulation stability were conducted immediately after preparation of SCH772984 encapsulated nanoparticles. Particle size analysis shows that the hydrodynamic diameter of the nanoparticles was 185±20 nm, Surface charge (ζ-potential) of the particles was found to be −3.35±0.10 mV.

Drug release studies showed that nanoparticles were able to respond to both hypoxia and acidified pH environment. Drug release kinetics followed Korsmeyer-Peppas model indicating polymer swelling was the major mechanism that contributed to drug release. After successful physical characterization, the in vitro antiproliferative activity of the combination therapy consists of nanoencapsulated ERKi with GEM was investigated in two types of pancreatic cancer cells, i.e., MIA PaCa-2 and PANC-1. Cytotoxicity assay of the drug combination elicited significant synergistic interactions against MIA PaCa-2 cell line more than for PANC-1 cells. The inhibition of colony formation and cellular motility of MIA PaCa-2 or PANC-1 was evaluated by treating the cells with unencapsulated GEM, ERKi or nanoparticle encapsulated ERKi+GEM combination. The highest efficiency to inhibit the colony formation was observed for nanoparticle encapsulated ERKi+GEM combination in comparison to all other treatment groups.

The pancreatic cancer cell migration was also suppressed significantly by the same drug combination compared to others. The mechanism of cell death was investigated by studying the hallmark experiment of apoptosis, measurement of mitochondrial membrane potential by the dual emission dye, JC1. The percentage of apoptotic cells were found to increase after treatment with the drug combination (nanoparticle encapsulated ERKi+GEM combination) in a time dependent manner. In summary, combination therapy of nanoparticle encapsulated ERKi and GEM deciphered efficient, in vitro therapeutic outcome against PDAC cells.

Features and advantages of this disclosure are apparent from the detailed specification, and the claims cover all such features and advantages. Numerous variations will occur to those skilled in the art, and any variations equivalent to those described in this disclosure fall within the scope of this disclosure. Those skilled in the art will appreciate that the conception upon which this disclosure is based may be used as a basis for designing other methods and systems for carrying out the several purposes of this disclosure. As a result, the claims should not be considered as limited by the description or examples.

What is claimed is:

1. A composition comprising a nanoparticle having a core comprising an inhibitor of extracellular signal-regulated kinase 1 or 2 (ERK1 or ERK2) and a polymer carrier, wherein the polymer carrier encapsulates the inhibitor, wherein the polymer carrier comprises a copolymer of poly(ethylene glycol) (PEG) and a poly(carbonate), and wherein the copolymer comprises a pH-responsive ligand that facilitates release of the inhibitor from the nanoparticle when the nanoparticle is present in an acidic or hypoxic environment.

2. The composition of claim 1, wherein the inhibitor is a compound represented by the formula:

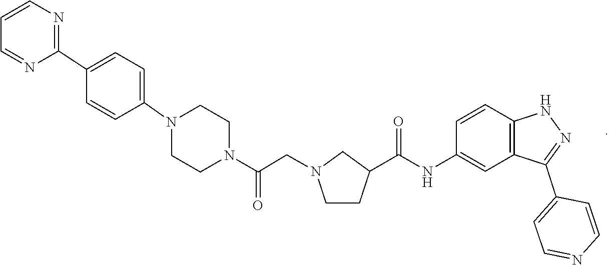 [SMILES: O=C(Nc1ccc2[nH]nc(-c3ccncc3)c2c1)C1CCN(CC(=O)N2CCN(c3ccc(-c4ncccn4)cc3)CC2)C1 ; "."]

3. The composition of claim 1, further comprising a chemotherapeutic agent.

4. The composition of claim 3, wherein the chemotherapeutic agent is a nucleoside analog.

5. The composition of claim 4, wherein the nucleoside analog is a pyrimidine nucleoside prodrug.

6. The composition of claim 3, wherein the chemotherapeutic agent is gemcitabine.

7. The composition of claim 3, wherein the chemotherapeutic agent is encapsulated by a polymer carrier of a nanoparticle having a core comprising the chemotherapeutic agent, wherein the polymer carrier is a copolymer of poly(ethylene glycol) (PEG) and a poly(carbonate), and wherein the copolymer comprises a pH-responsive ligand that facilitates release of the inhibitor from the nanoparticle when the nanoparticle is present in an acidic or hypoxic environment.

8. The composition of claim 3, wherein the core of the nanoparticle comprises the chemotherapeutic agent, wherein the polymer carrier encapsulates the chemotherapeutic agent, and wherein the pH-responsive ligand facilitates the release of the chemotherapeutic agent from the nanoparticle when the nanoparticle is present in an acidic or hypoxic environment.

9. The composition of claim 1, wherein the nanoparticle has an average particle size of less than 200 nm.

10. The composition of claim 1, wherein the copolymer is a block copolymer of poly(ethylene glycol) (PEG) and the poly(carbonate).

11. The composition of claim 1, wherein the copolymer comprises a ligand that targets a pancreatic cancer cell.

12. The composition of claim 11, wherein the ligand targets a pancreatic ductal adenocarcinoma (PDAC) cell.

13. The composition of claim 11, wherein the ligand comprises an arginine-glycine-aspartate (RGD) residue.

14. The composition of claim 11, wherein the ligand comprises iRGD.

15. The composition of claim 1, wherein the copolymer has a structure represented by the formula: 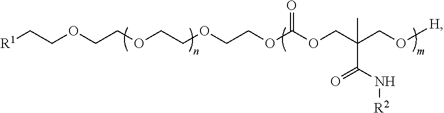
wherein R¹ is C₁-C₄ alkoxy or a ligand that targets a pancreatic cancer cell, R² is C₁-C₁₂ alkyl, C₁-C₁₂ alkylamino, C₂-C₁₂ dialkylamino, C₃-C₁₂ cycloalkyl, or C₂-C₁₂ heterocycloalkyl, and n and m are independently integers ranging from 10 to 300.

16. The composition of claim 15, wherein the copolymer has a structure represented by the formula: 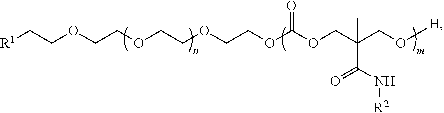
wherein R¹ is a ligand that targets a pancreatic cancer cell, having a structure represented by the formula:

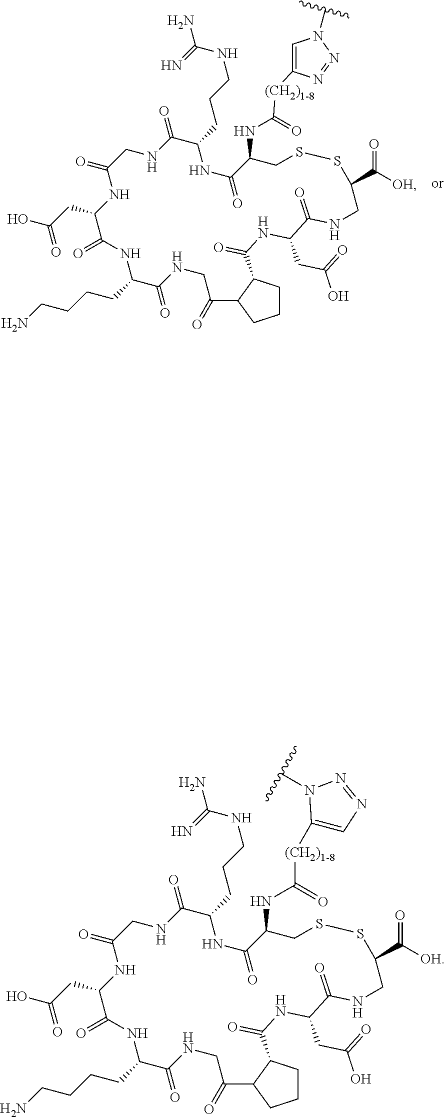 [SMILES: *n1cc(CC(=O)N[C@H]2CSS[C@@H](C(=O)O)CNC(=O)[C@H](CC(=O)O)NC(=O)[C@@H]3CCCC3C(=O)CNC(=O)[C@H](CCCCN)NC(=O)[C@H](CC(=O)O)NC(=O)CNC(=O)[C@H](CCCNC(=N)N)NC2=O)nn1 ; "or" ; *n1nncc1CC(=O)N[C@H]1CSS[C@@H](C(=O)O)CNC(=O)[C@H](CC(=O)O)NC(=O)[C@@H]2CCCC2C(=O)CNC(=O)[C@H](CCCCN)NC(=O)[C@H](CC(=O)O)NC(=O)CNC(=O)[C@H](CCCNC(=N)N)NC1=O]

17. A method of treating a pancreatic cancer in a subject in need thereof, comprising administering to the subject a composition comprising:
a) a nanoparticle having a core comprising an inhibitor of extracellular signal-regulated kinase 1 or 2 (ERK1 or ERK2) and a polymer carrier, wherein the polymer carrier encapsulates the inhibitor, wherein the polymer carrier is a copolymer of poly(ethylene glycol) (PEG) and a poly(carbonate), and wherein the copolymer comprises a pH-responsive ligand that facilitates release of the inhibitor from the nanoparticle when the nanoparticle is present in an acidic or hypoxic environment; and
b) a chemotherapeutic agent.

18. The method of claim 17, wherein the chemotherapeutic agent is gemcitabine.

19. The method of claim 17, wherein the chemotherapeutic agent is encapsulated by a polymer carrier of a nanoparticle having a core comprising the chemotherapeutic agent, wherein the polymer carrier is a copolymer of poly(ethylene glycol) (PEG) and poly(carbonate), and wherein the copolymer comprises a pH-responsive ligand that facilitates release of the inhibitor from the nanoparticle when the nanoparticle is present in an acidic or hypoxic environment.

20. The method of claim 17, wherein the polymer carrier encapsulates the chemotherapeutic agent, and wherein the pH-responsive ligand facilitates the release of the chemotherapeutic agent from the nanoparticle when the nanoparticle is present in an acidic or hypoxic environment.
